(19) United States
(12) Patent Application Publication
(10) Pub. No.: US 2025/0339403 A1
(43) Pub. Date: Nov. 6, 2025

(54) Tryptamine Formulations and Uses Thereof

(71) Applicant: Natural MedTech Pty Ltd, Sherbrooke (AU)

(72) Inventors: Sanjay Garg, Sherbrooke (AU); Yunmei Song, Sherbrooke (AU); Candace Minhthu Day, Sherbrooke (AU); Mark Heinrich Wolfgang Hestermann, Sherbrooke (AU); Luke Andrew McFarlane, Sherbrooke (AU)

(21) Appl. No.: 19/200,583

(22) Filed: May 6, 2025

Related U.S. Application Data
(63) Continuation of application No. PCT/AU2023/051122, filed on Nov. 7, 2023.

(30) Foreign Application Priority Data
Nov. 7, 2022 (AU) 2022903320

(51) Int. Cl. A61K 31/4045; A61K 31/5375; A61K 31/675

(52) U.S. Cl. CPC A61K 31/4045; A61K 31/5375; A61K 31/675

(57) ABSTRACT

The invention relates to formulations of tryptamines and monoamine oxidase inhibitors and uses thereof in methods of treatment. In particular, compositions having a modified-release of a tryptamine and an immediate release of a monoamine oxidase inhibitor. Also disclosed are methods of treating disorders with tryptamines in combination with monoamine oxidase inhibitors.

This application is a continuation of international application No. PCT/AU2023/051122, filed on Nov. 7, 2023, which is claiming priority from Australian Provisional Application No. 2022903320, filed on Nov. 7, 2022, the entire contents of each of which are incorporated herein by reference.

### TECHNICAL FIELD

The invention relates to formulations of tryptamines and monoamine oxidase inhibitors and uses thereof in methods of treatment. In particular, compositions having a modified-release of a tryptamine and an immediate release of a monoamine oxidase inhibitor. Also disclosed are methods of treating disorders with tryptamines in combination with monoamine oxidase inhibitors.

### BACKGROUND OF THE INVENTION

Serotonin has been identified as a critical neurotransmitter involved in mood modulation. In the 1960s, Harvard psychiatrist Joseph Schildkraut proposed the Serotonin Hypothesis of Depression. It postulated that a deficit in the production or secretion of serotonin within the central nervous system, particularly in regions such as the pre-frontal cortex, may be a critical causal factor in mood and anxiety disorders. As such, correction of this imbalance could address a range of affective disorders, including depression.

While this theory is largely considered as an oversimplification of complex physical and psychological factors which interact to influence mood, there has been widespread use in psychiatry of pharmaceuticals which influence serotonin signalling and abundance, such as selective serotonin reuptake inhibitors (SSRIs), for the treatment of mood disorders.

It is not yet fully understood how these psychotherapeutic interventions work, however evidence suggests that in addition to increases in synaptic serotonin, changes in brain structure and function accompany the therapeutic effects of these drugs. Clinical use also indicates that treatment which increases serotonin or serotonin signalling can increase personal meaningfulness, mindfulness, introspection and result in a positive change in the patient's outlook on life.

Despite significant initial research and development of drugs which alter the abundance of serotonin or serotonin signalling, there has been little recent development in this area.

Due to their mechanism of action, and significant anecdotal evidence from traditional and cultural use, the use of psychedelics in treatment of a range on neurological and psychological disorders and have begun to be studied. A large number of traditionally used psychedelics have been identified as potent agonists of serotonin 5-hydroxytryptamine (5-HT) receptors, and activation of these receptors has been identified as being essential for their psychedelic effects.

Psychedelic drugs typically fall under one of three classes; (1) the tryptamines, (2) the Phenethylamines (PEA), and (3) the lysergamides. Each of these classes (and compounds within these classes) have varied activity against both serotonin and dopamine receptors.

Of particular interest are psychedelic tryptamines. These are structural analogues for serotonin and have been shown to bind to 5-HT receptors, including 5-HT_{2A}. However, the use of these psychedelic tryptamines in a clinical setting is limited by a range of factors such as their bioavailability and possibilities for abuse.

Ayahuasca is a commonly used psychoactive preparation often consumed in parts of South America. Ayahuasca is known to contain psychedelic tryptamines (e.g. N,N-dimethyltryptamine, DMT) that act at 5-HT receptors and various harmala alkaloids (e.g. harmine and harmaline) that inhibit the action of monoamine oxidases, which are responsible for metabolism of tryptamines. While the desired hallucinogenic and/or mystical effects of ayahuasca are observed in those consuming the beverage, unwanted side effects such as nausea and vomiting are often experienced by the subject. Furthermore, the desired effects attributed to ayahuasca often requires repeated ingestion of the beverage. The traditional means for consumption of Ayahuasca, which is served as a tea prepared as a decoction of the bush Psychotria viridis and Banisteriopsis caapi, can be used to provide oral dosing of psychedelic tryptamines. The brew typically comprises a series of serotonergic agonists and reuptake inhibitors. Due to the means for preparing Ayahuasca, there can be significant variations in the dose and there can be significant variation in the psychoactive ingredients.

Where the administration of DMT, for example, is required, doses are typical smoked or inhaled. Other methods of administration include intravenous or intramuscular injection, however none of these methods are practical for delivery in a clinical (or non-clinical) setting.

There remains a need for new compositions and therapeutic and non-therapeutic methods for treatment by administration of tryptamines.

### SUMMARY OF THE INVENTION

To allow clinical use of tryptamines it is desired to provide a convenient dosage formulation. Particularly, a dosage formulation which can be consumed as a single form pharmaceutical and improves the bioavailability of the tryptamine. This is made particularly difficult due to the rapid systemic metabolism of many tryptamines when orally ingested. While alternative routes of administration exist, such as intramuscular injection and vapor inhalation, these are not amenable to routine clinical use.

Further, the supply of tryptamines in an unmodified form may be susceptible to abuse. Indeed, in most jurisdictions, psychedelic tryptamines are prohibited substances and therefore the provision of psychedelic tryptamines in a form which has a reduced potential for abuse is also desirable.

Furthermore, to assist in providing clinical use of psychedelic tryptamines, it may be advantageous that any noticeable perceptual effects (such as hallucinogenic effects) are time-limited to allow clinical supervision within standard clinical rotations.

Most psychedelic tryptamines are immediately absorbed into the body where they are rapidly degraded by monoamine oxidase. This means that many tryptamines are poorly orally bioavailable, due to first pass metabolism. Furthermore, their time above a therapeutic, or other desired threshold, can be significantly time limited due to rapid metabolism. Accordingly, the present invention provides a composition comprising a monoamine oxidase inhibitor (MAOI) and a tryptamine. The present inventors believe that the administration of a MAOI can be used to regulate the peak plasma concentration of the tryptamine and subsequently the duration for which the plasma concentration of the tryptamine is above a given threshold level of the plasma concentration of the psychedelic tryptamine. Administration of a MAOI prior to administration of the tryptamine means that the activity of the MAO enzyme is reduced or inhibited, such that the tryptamine (once administered) is able to act at 5-HT receptors instead of being oxidised by the enzyme.

By combining the two components into a single dosage form, this allows for a simplified and single administration of the composition. This assists in utilising the composition in a clinical setting because it means that patient compliance is improved, variations in timing are decreased, and clinical supervision can be provided (when needed) at the time of dosing with the tryptamine.

In a first aspect, the present invention provides a method for the treatment of a neurodevelopmental, neurological, movement, psychological or physiological, disease, disorder, syndrome or symptom in a subject, the method comprising administering a monoamine oxidase inhibitor (MAOI) and a tryptamine, derivative thereof or pharmaceutically acceptable salt thereof, wherein the MAOI is formulated for immediate release and the tryptamine is formulated for delayed release, wherein the tryptamine is a substrate for a MAO.

In a second aspect, the present invention also provides a method of enhancing the effect of a tryptamine in a subject, the method comprising administering a monoamine oxidase inhibitor (MAOI) and a tryptamine, derivative thereof or pharmaceutically acceptable salt thereof, wherein the MAOI is formulated for immediate release and the tryptamine is formulated for delayed release, wherein the tryptamine is a substrate for a MAO.

In certain embodiments, the tryptamine is a psychedelic tryptamine.

In some preferred embodiments of the present invention, the psychedelic tryptamine is selected from the group consisting of: psilocin, psilocybin, N,N-dimethyltryptamine (DMT), 5-methoxy-N,N-dimethyltryptamine (5-MeO-DMT), or a pharmaceutically acceptable salt or prodrug thereof. Most preferred are DMT, psilocin, psylocibin and pharmaceutically acceptable salt or prodrugs thereof.

In certain embodiments, the MAOI is a reversible inhibitor. In other embodiments, the MAOI is a selective monoamine oxidase A (MAO-A) inhibitor. In other embodiments, the MAOI is a reversible inhibitor selective for MAO-A. In other embodiments, the MAOI is a selective and reversible inhibitor of monoamine oxidase A (RIMA). In other embodiments, the MAOI is a selective and non-reversible inhibitor of monoamine oxidase A.

In some embodiments, the MAOI is moclobemide.

In some embodiments, the MAOI and the tryptamine are administered as separate dosage forms. In other embodiments, the MAOI and the tryptamine are administered in the same dosage form.

Typically, monoamine oxidase inhibitors will have a delay between dosage and inhibition of monoamine oxidase. Therefore, it is advantageous to expedite the absorption of the MAOI to allow as rapid inhibition of the monoamine oxidase, while at the same time offsetting (i.e., delaying) the absorption of the tryptamine to allow sufficient inhibition of the monoamine oxidase prior to release of the tryptamine. Therefore, in some embodiments, the single dosage form pharmaceutical comprises an immediate-release formulation of the MAOI and a modified-release (preferably delayed-release) formulation of the tryptamine.

In a third aspect, the present invention provides a method of treating a neurodevelopmental, neurological, movement or psychological, disease, disorder or syndrome in a subject, the method comprising administering a single dosage form composition comprising a monoamine oxidase inhibitor (MAOI) and a tryptamine, derivative thereof or pharmaceutically acceptable salt thereof, wherein the MAOI is formulated for immediate release and the tryptamine is formulated for delayed release, wherein the tryptamine is a substrate for a MAO.

In some embodiments, the release of the MAOI is complete within about 1 hour. In other embodiments, the release of the MAOI is complete within about 30 minutes.

In certain embodiments, the release of the tryptamine occurs over a time of up to about 12 hours after administration. In some embodiments, release of the tryptamine occurs over a time of up to about 10 hours after administration. In other embodiments, release of the tryptamine occurs over a time of up to about 8 hours after administration. In some embodiments, about 20% of the tryptamine is released within about 3 hours after administration. In some embodiments, about 20% of the tryptamine is released between about 1 and about 3 hours after administration. In other embodiments, about 20% of the tryptamine is released within about 2 hours after administration. In other embodiments, about 20% of the tryptamine is released within about 1 hour after administration. In yet other embodiments, about 20% of the tryptamine is released in less than 1 hour after administration. In other embodiments, about 20% of the tryptamine is released in less than 45 minutes after administration. IN further embodiments, about 20% of the tryptamine is released in less than 30 minutes after administration. In some embodiments, about 50% of the tryptamine is released between about 2 and about 6 hours after administration. In some embodiments, about 50% of the tryptamine is released between about 2 hours and about 4 hours after administration. In other embodiments, about 90% of the tryptamine is released between about 6 and about 12 hours after administration. In other embodiments, about 90% of the tryptamine is released between about 6 and about 10 hours after administration. In other embodiments, about 90% of the tryptamine is released between about 6 and about 8 hours after administration. In other embodiments, about 90% of the tryptamine is released between about 8 hours and about 10 hours after administration.

In some embodiments, between about 20% to about 90% of the tryptamine that is administered is released between about 2 and about 10 hours after administration.

While the release of the tryptamine may be formulated to occur over a number of hours, the present inventors have also found that compositions having a bolus release of tryptamine may also be prepared, where a large proportion of the tryptamine present in the composition is released in a short time after administration to a subject. In certain embodiments, the composition comprises tryptamine or a pharmaceutically acceptable salt thereof, wherein about 70% to about 90% of the tryptamine is released within about 15 minutes after administration to a subject. In certain embodiments, release of about 70% of the tryptamine occurs within about 15 minutes after administration to the subject. In certain embodiments, release of about 75% of the tryptamine occurs within about 15 minutes after administration to the subject. In certain embodiments, release of about 80% of the tryptamine occurs within about 15 minutes after administration to the subject. In other embodiments, release of about 85% of the tryptamine occurs within about 15 minutes of administration to the subject. In certain embodiments, release of about 90% of the tryptamine occurs within about 15 minutes after administration to the subject.

In order to provide the release properties discussed above, the present inventors have found that a composition comprising a MAOI and a tryptamine may be produced such that the MAOI is formulated for immediate release and the tryptamine is formulated for delayed release.

In a fourth aspect, the present invention provides a composition comprising a monoamine oxidase inhibitor (MAOI) and a tryptamine, wherein the MAOI is formulated for immediate release and the psychedelic tryptamine is formulated for delayed release, wherein the tryptamine is a substrate for a MAO. In certain embodiments, the composition comprising a MAOI for immediate release and a tryptamine for delayed release is provided as a single dosage form.

While the compositions comprising a MAOI and a tryptamine of the present invention can take any suitable form, in some preferred embodiments, the dosage form is a solid dosage form. This allows simple packaging, storage, distribution, dispensing and administration.

The compositions disclosed herein can be of any suitable constructions. However, in some embodiments it comprises a multilayer tablet with a first layer comprising an immediate-release MAOI and a second layer comprising a modified-release tryptamine. In some alternative embodiments, it comprises a tablet comprising an immediate-release MAOI outer and a modified-release tryptamine core. In some further embodiments, it comprises a capsule comprising at least one immediate-release bead containing a MAOI and at least one modified-release bead containing the tryptamine.

While in some embodiments, the composition is formulated for enteral administration, in some embodiments, it may be formulated for sublingual or buccal administration and therefore may take be in the form of a sublingual or buccal composition. Envisaged sublingual or buccal compositions include a film or a tablet.

In certain embodiments, the composition comprises a monoamine oxidase inhibitor (MAOI), a tryptamine, and a pharmaceutically acceptable excipient, carrier or stabiliser.

In some embodiments of the compositions disclosed herein, the tryptamine is a psychedelic tryptamine. In some embodiments of the compositions disclosed herein, the psychedelic tryptamine is selected from the group consisting of psilocin, psilocybin, N,N-dimethyltryptamine (DMT), 5-methoxy-N,N-dimethyltryptamine (5-MeO-DMT), and pharmaceutically acceptable salts and prodrugs thereof.

In some embodiments, the composition is in a solid form.

In some embodiments, the composition is formulated for immediate release of the MAOI. Accordingly, in some embodiments, the composition is formulated for sequential release of the MAOI followed by the tryptamine.

In some embodiments, the composition is an oral composition or is for use as an oral composition. In some embodiments, the oral composition is in the form of a tablet or capsule. In other embodiments, the composition is a powder. In other embodiments, the composition is in the form of a gel. In other embodiments, the composition is in the form of a film.

In some embodiments, the composition is an enteral composition or is for use as an enteral composition. In some alternative embodiments, the composition is a sublingual or buccal composition. The sublingual or buccal composition can take any form, but in some embodiments, it is in the form of a tablet or a film.

As indicated above, in some forms of the invention the MAOI is formulated for immediate release. Accordingly, this may result in embodiments of the invention which are formulated for complete release of the MAOI within 2 minutes, within 5 minutes, within 10 minutes, within 15 minutes, within 20 minutes, within 25 minutes, or within 30 minutes after administration to a subject. In certain embodiments, complete release of the MAOI occurs within about 1 hour after administration to a subject.

In a fifth aspect, the present invention provides a composition comprising a monoamine oxidase inhibitor (MAOI) and one or more excipients, wherein the MAOI is formulated for immediate release.

To provide sequential release of the psychedelic tryptamine after the MAOI, the composition is formulated in some embodiments such that release of the psychedelic is delayed for at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 120 minutes, at least 130 minutes, at least 140 minutes, at least 150 minutes, at least 200 minutes, at least 250 minutes or at least 300 minutes after administration of the composition to a subject.

To allow sufficient inhibition of the monoamine oxidase prior to release of the tryptamine, and therefore allow sufficient bioavailability of the tryptamine, it is advantageous to delay the time to peak concentration of the tryptamine after administration of a composition comprising the tryptamine. Therefore, in some embodiments, the composition is formulated to produce a median time to peak concentration of the tryptamine from 15 minutes, 30 minutes, 60 minutes, 90 minutes to 150 minutes, from 100 minutes to 140 minutes, or from 110 minutes to 130 minutes, or from 115 minutes to 125 minutes or approximately 120 minutes after administration to a subject. The present inventors have shown that the administration of a composition comprising a MAOI that is formulated to be released immediately can delay the peak concentration of a tryptamine that is administered at the same time.

Monoamine oxidase inhibitors known in the art include the group consisting of 1,2,3,4-tetrahydro-ß-carboline, 1,2,3,4-tetrahydroisoquinoline (TIQ), 2-methyl-1,2,3,4-tetrahydro-ß-carboline, Amiflamine, Befloxatone, Benmoxin, Bifemelane, Brofaromine, Caroxazone, Clorgyline, Curcumin, CX157 (TriRima), Epicatechin, Eprobemide, Esuprone, Harmaline, Harmane Harmine, Hydracarbazine, Iproclozide, Iproniazid, Isocarboxazid, Mebanazine, Methylene blue, Methylthioninium chloride, Metralindole, Minaprine, Moclobemide, Nialamide, Norharmane, Octamoxin, Pargyline, Phenelzine, Pheniprazine, Phenoxypropazine, Pirlindole, Pivalylbenzhydrazine, Procabazine, Procyanidins, Quercetin, Rasagiline, Rosiridin, Safinamide, Safrazine, Sercloremine, Selegiline, Tetrahydroharmine, Tetrindole, Toloxatone, and Tranylcypromine.

Preferred MAOI include the group consisting of: Bifemelane, Hydracarbazine, Iproniazid, Isocarboxazid, Methylthioninium chloride, Moclobemide, Phenelzine, Pirlindole, Tranylcypromine, Rasagiline, Selegiline, and Safinamide. All of these MAOI have been approved by a pharmaceutical regulatory body somewhere in the world. The most preferred MAOI is Moclobemide, which has the advantage of being reversible and selective for MAO-A.

Accordingly, in some embodiments, the composition comprises up to 10 mg, up to 25 mg, up to 50 mg, up to 100 mg, up to 150 mg, up to 200 mg, up to 250 mg, up to 300 mg, up to 350 mg, up to 400 mg, up to 450 mg, up to 500 mg, up to 550 mg, up to 600 mg, up to 700 mg, up to 800 mg, up to 900 mg, up to 1000 mg, up to 1100 mg, or up to 1200 mg of a MAOI. In certain embodiments, the MAOI is Moclobemide.

As disclosed herein, the present inventors have found that a composition comprising a tryptamine that is formulated in accordance with embodiments of the invention provide a delay in the time to reach peak concentration after administration to a subject.

In a sixth aspect, the present invention provides a composition comprising a tryptamine and one or more excipients, wherein the tryptamine is formulated for delayed release and wherein the tryptamine is a substrate for a MAO.

In certain embodiments, the tryptamine is a psychedelic tryptamine.

In certain embodiments, the psychedelic tryptamine is selected from the group consisting of psilocin, a prodrug of psilocin, psilocybin, N,N-dimethyltryptamine (DMT), 5-methoxy-N,N-dimethyltryptamine (5-MeO-DMT) and pharmaceutically acceptable salts and prodrugs thereof.

In some embodiments, the psychedelic tryptamine is DMT, or a pharmaceutically acceptable salt or prodrug thereof. N,N-dimethyltryptamine is very rapidly metabolised by peripheral monoamine oxidases (particularly MAO-A) and therefore is not regarded as orally bioavailable due to significant first-pass metabolism. N,N-dimethyltryptamine is available as a variety of pharmaceutically available salts, as well as in a free-base form. However, in some embodiments, the DMT is provided as DMT fumarate.

In certain embodiments, release of the tryptamine is delayed for between about 5 minutes and about 3 hours. In another embodiment, release of the tryptamine is delayed for about 2 hours.

The present inventors have found that the delayed release properties of a composition comprising a tryptamine as disclosed herein are attributed to the one or more excipients present in the composition. Without wishing to be bound by theory, the present inventors believe that the release properties of the tryptamine as disclosed herein result from the selection of polymers that are used to provide a coating to the psychedelic tryptamine. The compositions comprising a tryptamine are formulated such that release of the active component is delayed for a time, however release of the active is also achieved over a certain period of time. The present inventors have found that the use of one or more polymers in a coating for the tryptamine as disclosed herein provide the requisite release properties while also being compatible with the tryptamine.

In some embodiments, the present invention provides a dose of a tryptamine which does not result in noticeable perceptual effects, such as hallucinations. Therefore, in some embodiments composition is formulated to provide a peak plasma concentration of the tryptamine in a subject after administration below a maximum threshold of up to 10 μg/L, up to 11 μg/L, up to 12 μg/L, up to 13 μg/L, up to 14 μg/L, up to 15 μg/L, up to 16 μg/L, up to 17 μg/L, up to 18 μg/L, up to 19 μg/L, up to 20 μg/L, up to 25 μg/L, up to 30 μg/L, up to 35 μg/L, up to 40 μg/L, up to 45 μg/L, up to 50 μg/L, up to 55 μg/L, up to 60 μg/L, up to 65 μg/L, up to 70 μg/L, up to 100 μg/L, up to 150 μg/L, up to 200 μg/L, up to 250 μg/L, or up to 300 g/L.

It is also desirable (in some embodiments) to provide a dose of a tryptamine which results in a plasma concentration above a therapeutic threshold, such as a threshold where there is a psychotropic or serotonergic effect. Accordingly, in some embodiments, the composition is formulated to provide a plasma concentration of a tryptamine in a subject after administration above a therapeutic threshold level of at least 1 μg/L, at least 2 μg/L, at least 3 μg/L, at least 4 μg/L, at least 5 μg/L, at least 6 μg/L, at least 7 μg/L, at least 8 μg/L, at least 9 μg/L, at least 10 μg/L, at least 11 μg/L, at least 12 μg/L, at least 13 μg/L, at least 14 μg/L, at least 16 μg/L, at least 18 μg/L, or at least 20 μg/L.

It is further desirable to not just reach a plasma level above a therapeutic threshold, but to also prolong the time above this threshold to improve the therapeutic effects. Therefore, in some embodiments the composition is formulated to provide a plasma concentration of a tryptamine in a subject after administration above the therapeutic threshold for at least 30 minutes, at least 60 minutes, at least 90 minutes, at least 120 minutes, at least 150 minutes, at least 180 minutes, at least 210 minutes, at least at least 240 minutes, at least 270 minutes, at least 300 minutes, and least 330 minutes, at least 360 minutes, at least 390 minutes, at least 420 minutes, at least 450 minutes, at least 480 minutes, at least 510 minutes, at least 540 minutes, or at least 570 minutes.

In certain embodiments, a composition comprises a tryptamine or a pharmaceutically acceptable salt thereof in an amount of up to 2 mg, 3 mg, 4 mg, 5 mg, up to 6 mg, up to 7 mg, up to 8 mg, up to 9 mg, up to 10 mg, up to 11 mg, up to 12 mg, up to 13 mg, up to 14 mg, up to 15 mg, up to 20 mg, up to 25 mg, up to 30 mg, up to 35 mg, up to 40 mg, up to 50 mg, up to 60 mg, up to 70 mg, up to 80 mg, up to 100 mg, up to 150 mg, up to 200 mg, up to 250 mg or up to 300 mg.

In certain embodiments, a composition as disclosed herein is formulated to provide a plasma concentration of a tryptamine in a subject after administration above a threshold level of at least 12 μg/L, at least 15 μg/L, at least 20 μg/L, at least 25 μg/L, at least 30 μg/L, at least 35 μg/L, at least 40 μg/L, at least 45 μg/L, at least 50 μg/L, at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, at least 100 μg/L, at least 150 μg/L, at least 200 μg/L, at least 250 μg/L or at least 300 μg/L.

Another preferred tryptamine for use in a composition of the present invention is psilocin, a prodrug of psilocin, or a pharmaceutically acceptable salt thereof. In an embodiment, the prodrug of psilocin is psilocybin.

In some embodiments, the composition of the present invention comprises up to 1 mg, up to 2 mg, up to 3 mg, up to 4 mg or up to 6 mg of psilocin or the prodrug equivalent of psilocin, or a pharmaceutically acceptable salt thereof.

When using psilocin, as with DMT, it may be advantageous to not induce noticeable perceptual effects (such as hallucinogenic effects). Accordingly, in some embodiments, the composition of the present invention is formulated to provide a peak plasma concentration of psilocin in a subject after administration below a maximum threshold of up to 1 μg/L, up to 2 μg/L, up to 3 μg/L, up to 4 μg/L, up to 5 μg/L, up to 6 μg/L, up to 7 μg/L, up to 8 μg/L, up to 9 μg/L or up to 10 μg/L.

Further, it may be advantageous to provide a plasma concentration of psilocin above a therapeutic threshold, such as a psychotropic or serotonergic threshold. Therefore, in some embodiments, there is provided a composition, formulated to provide a plasma concentration of psilocin in a subject after administration above a therapeutic threshold of at least 0.2 μg/L, at least 0.3 μg/L, at least 0.4 μg/L, at least 0.5 μg/L, at least 0.6 μg/L, at least 0.7 μg/L, at least 0.8 μg/L, at least 0.9 μg/L, at least 1 μg/L, at least 2 μg/L, at least 5 μg/L, at least 10 μg/L, at least 12 μg/L, at least 14 μg/L, at least 16 μg/L, at least 18 μg/L, or at least 20 μg/L.

In some embodiments, the composition is formulated to provide a plasma concentration of psilocin in a subject after administration above a therapeutic threshold of at least 0.2 μg/L and up to 25 μg/L.

In some embodiments the composition is formulated to provide a plasma concentration of psilocin in a subject after administration above the therapeutic threshold for at least 60, at least 120, at least 180, at least 240, at least 300, at least 360, at least 420, at least 480, at least 540, at least 600, at least 660, at least 720, at least 780, at least 840, at least 900 minutes.

Also provided by the present invention is a method of treating a neurodevelopmental, neurological, movement, psychological or physiological, disease, disorder, syndrome or symptom in a subject including the steps of administering a single dosage form composition comprising a monoamine oxidase inhibitor (MAOI) and a tryptamine, wherein the tryptamine is a substrate for a MAO.

Further, there is provided a method of enhancing the effect of a psychedelic tryptamine in a subject including the steps of administering a single dosage form composition comprising a monoamine oxidase inhibitor (MAOI) and a tryptamine, wherein the tryptamine is a substrate for a MAO.

In some embodiments of the methods disclosed herein, the tryptamine is a psychedelic tryptamine. In some embodiments, the psychedelic tryptamine is selected from the group consisting of: psilocin, psylocibin, N,N-dimethyltryptamine (DMT), 5-methoxy-N,N-dimethyltryptamine (5-MeO-DMT), or a prodrug thereof, or a pharmaceutically acceptable salt thereof. Preferably, the psychedelic tryptamine is DMT, psilocybin or psilocin, a prodrug thereof, or a pharmaceutically acceptable salt thereof.

In some embodiments, the enhanced effect of the psychedelic is a hallucinogenic effect. In other embodiments, the enhanced effect is a therapeutic effect.

Is some embodiments, the therapeutic effect is treating a neurodevelopmental, neurological, movement, psychological or physiological, disease, disorder, syndrome or symptom.

In some embodiments of the method, the composition is administered to provide a dose of a tryptamine up to 4 mg/kg, up to 2 mg/kg, up to 1 mg/kg, up to 0.5 mg/kg, up to 0.3 mg/kg, up to 0.25 mg/kg, up to 0.2 mg/kg, up to 0.15 mg/kg, up to 0.1 mg/kg, or up to 0.05 mg/kg. In certain embodiments, the tryptamine is a psychedelic tryptamine. In other embodiments, the psychedelic tryptamine is DMT or psilocybin.

In some embodiments, the methods comprise maintaining the peak plasma concentration of a tryptamine in a subject after administration of the composition below a maximum threshold of up to 10 μg/L, up to 11 μg/L, up to 12 μg/L, up to 13 μg/L, up to 14 μg/L, up to 15 μg/L, up to 16 μg/L, up to 17 μg/L, up to 18 μg/L, up to 19 μg/L, up to 20 μg/L, up to 25 μg/L, up to 30 μg/L, up to 50 μg/L, up to 100 μg/L, up to 150 μg/L, up to 200 μg/L, up to 250 μg/L, or up to 300 μg/L.

In some embodiments, the methods result in a plasma concentration of a tryptamine above a therapeutic threshold of at least 0.5 μg/L, at least 1 μg/L, at least 2 μg/L, at least 3 μg/L, at least 4 μg/L, at least 5 μg/L, at least 6 μg/L, at least 7 μg/L, at least 8 μg/L, at least 9 μg/L, at least 10 μg/L, at least 11 μg/L, at least 12 μg/L, at least 13 μg/L, or at least 14 μg/L.

In some embodiments, the methods comprise maintaining the plasma concentration of the tryptamine above the therapeutic threshold for at least 15 minutes, at least 30 minutes, at least 60 minutes, at least 120 minutes, at least 150 minutes, at least 180 minutes, at least 210 minutes, at least 240 minutes, at least 270 minutes, at least 300 minutes, at least 330 minutes, at least 360 minutes, at least 390 minutes, at least 420 minutes, at least 450 minutes, at least 480 minutes, at least 510 minutes, at least 540 minutes, at least 570 minutes, or at least 600 minutes.

In some embodiments, the methods comprise maintaining the plasma concentration of the tryptamine in a subject after administration of the single dosage form pharmaceutical or the pharmaceutical composition above a threshold level of at least 12 μg/L, at least 15 μg/L, at least 20 μg/L, at least 25 μg/L, at least 30 μg/L, at least 35 μg/L, at least 40 μg/L, at least 45 μg/L, at least 50 μg/L, at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, at least 100 μg/L, at least 150 μg/L, at least 200 μg/L or at least 250 μg/L. Preferably, the threshold level is the level where a subject experiences noticeable perceptual effects, such as hallucinations.

In some embodiments, the methods comprise maintaining the plasma concentration of a tryptamine above a threshold level for up to 15 minutes, up to 30 minutes, up to 35 minutes, up to 45 minutes, up to 50 minutes, up to 55 minutes, up to 60 minutes, or up to 90 minutes.

In some embodiments of the methods, the tryptamine is a psychedelic tryptamine. In other embodiments, the psychedelic tryptamine is psilocybin, psilocin or a prodrug of thereof.

In some embodiments of the methods, the composition is administered at a dose of psilocybin or psilocin up to 0.2 mg, up to 1 mg, up to 2 mg, up to 3, mg, up to 4 mg, up to 6 mg, up to 8 mg, up to 10 mg, up to 15 mg, up to 20 mg, up to 25 mg, up to 30 mg, up to 35 mg or up to 40 mg or a pro-drug equivalent thereof.

In some embodiments, the methods comprise maintaining the peak plasma concentration of psilocin in the subject after administration below a maximum threshold level of up to 1 μg/L, up to 2 μg/L, up to 3 μg/L, up to 4 μg/L, up to 5 μg/L, up to 6 μg/L, up to 8 μg/L, up to 10 μg/L, up to 12 μg/L, up to 14 μg/L, up to 16 μg/L, up to 18 μg/L, up to 20 μg/L up to 25 μg/L, up to 30 μg/L or up to 35 μg/L.

In some embodiments, the methods result in a plasma concentration of psilocin in the subject above a therapeutic threshold level of at least 0.2 μg/L to up to 25 μg/L.

In some embodiments, the methods comprise maintaining the plasma concentration of psilocin above the therapeutic threshold for at least 15, 30, 60, 120, 180, 240, 300, 360, 420, 480, 540, 600, 660, 720, 780, 840, or 900 minutes.

In a seventh aspect, there is provided the use of a MAOI formulated for immediate release and a tryptamine formulated for delayed release in the manufacture of a medicament for the treatment of a neurodevelopmental, neurological, movement, psychological or physiological, disease, disorder or syndrome in a subject, wherein the tryptamine is a substrate for a MAO.

In some embodiments, the tryptamine is a psychedelic tryptamine.

In an eighth aspect, the present invention provides a process for preparing a composition comprising a tryptamine formulated for delayed release, wherein an aqueous suspension comprising two or more polymers is atomized and applied to the surface of the tryptamine.

In a ninth aspect, the present invention provides a process for preparing a composition comprising a monoamine oxidase inhibitor (MAOI) for immediate release, the process comprising the steps of:
i) combining the MAOI, one or more disintegrants and a sugar;
ii) shaking the mixture of step i) and sieving the mixture of step i);
iii) adding an amount of water to the sieved mixture of step ii); and
iv) and extruding the mixture of step iii) to form the composition.

### BRIEF DESCRIPTION OF THE FIGURES

For a further understanding of the aspects and advantages of the present invention, reference should be made to the following detailed description, taken in conjunction with the accompanying drawings.

FIG. 1. Chart showing the percentage of DMT fumarate released from compositions formulated for delayed release, where the relative amounts of the coating applied to the DMT fumarate range from 60% to 160%. The amounts of the coating are calculated on based on the weight of the tablet composition, e.g. the 60% WG formulation refers to a tablet that has a coating weight of 60% of the tablet weight.

FIG. 2. Chart showing the percentage of DMT fumarate released from a composition comprising DMT fumarate, where the amount of coating is 40% of the composition weight. A delayed release composition of DMT fumarate with a 40% weight coating results in complete release of the DMT fumarate by 10 hours after administration, with about 60% of the DMT fumarate released within about 3 hours. This chart clearly shows that the rate of release is faster over the first 3 hours, compared to the following 7 hours.

FIGS. 3A-C. HPLC chromatograms of FIG. 3A a delayed release composition of DMT fumarate, FIG. 3B an immediate release composition of moclobemide, and FIG. 3C a composition comprising DMT fumarate formulated for delayed release and moclobemide formulated for immediate release. The chromatogram FIG. 3C of the composition comprising both active ingredients show that the analysis of a single dosage form comprising both active ingredients is possible given the baseline separation of peaks.

FIG. 4. Chart showing the release profile of a composition comprising both DMT fumarate formulated for delayed release and moclobemide formulated for immediate release. Dissolution of the composition comprising both active ingredients formulated accordingly show that release of moclobemide is complete within 1 hour of administration, while release of DMT fumarate occurs over at least 10 hours, with about 50% of the DMT fumarate released about 3 hours after administration.

FIG. 5. Picture of a capsule containing DMT fumarate formulated for delayed release and moclobemide formulated for immediate release. The capsule contains discrete particles of each of the active ingredients formulated accordingly.

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

Unless defined otherwise, all technical and scientific terms used herein have the same meaning as commonly understood by those of ordinary skill in the art to which the invention belongs. For the purposes of the present invention, the following terms are defined below.

The present invention relates to a method for the treatment of a neurodevelopmental, neurological, movement or psychological, disease, disorder or syndrome in a subject, the method comprising administering a monoamine oxidase inhibitor (MAOI) and a tryptamine, wherein the MAOI is formulated for immediate release and the tryptamine is formulated for delayed release, wherein the tryptamine is a substrate for a MAO. In certain embodiments, the tryptamine is a psychedelic tryptamine.

In some embodiments of the above method, the method of treatment includes providing psychotherapy or occupational therapy. In such an embodiment, the administration of the composition augments the therapy.

As used herein, the term “hallucinations” relates to a false perception or sensory perception occurring in a subject without external stimulation. For example, seeing, hearing, tasting, smelling or feeling things that are not there. Furthermore, hallucinations can include synesthesia.

As used herein, the term “perceptual effects” relates to any effect which is described as abnormal by a subject. These may include (but are not limited to) feelings of happiness or elation; changes in perception of speed or time; feelings of understanding; feelings of loss of control; feeling of, or connections to, spirituality or higher powers; feelings of mindfulness; feelings of fatigue or excitation; feelings of altered consciousness; altered perception of thoughts; loss of visual focus; feelings of disembodiment; inability to make decisions; hallucinations; feelings of altered understandings; feelings of power or empowerment; fear; changes in locomotion; changes in temporal perception; emotional engagement with objects; perception of enhanced beauty; out-of-body experiences; disconnected thoughts; new insights; uncontrolled or excessive humour; changes in auditory volume; changes in recollection; and euphoria. The term can further be defined by a person skilled in the art.

Also provided is a method of enhancing the effect of a tryptamine in a subject including the steps of administering a single dosage form pharmaceutical or a pharmaceutical composition comprising a monoamine oxidase inhibitor (MAOI) and a tryptamine, wherein the tryptamine is a substrate for a MAO. In certain embodiments, the tryptamine is a psychedelic tryptamine.

In some embodiments of the above method, the method of treatment includes providing psychotherapy or occupational therapy. In such an embodiment, the administration of the composition augments the therapy.

In some embodiments, it is desirable to provide a dose of the tryptamine below a maximum threshold level which induces noticeable perceptual effects. Such treatments may be referred to as “micro-dosing”) or sub-hallucinogenic dosing. Such sub-hallucinogenic dosing has been shown to have positive effects on cognitive processes as well as demonstrable effects on regions in the brain which are involved in affective processes. Accordingly, in some embodiments of the methods of the invention, the composition includes a dose of the tryptamine below a dose required to induce noticeable perceptual effects, such as hallucinations. However, in these embodiments, it may be desirable to induce a plasma concentration in a subject about a therapeutic level, such as a level whereby these are serotonergic and/or psychotropic effects.

References to a plasma concentration of an active ingredient as used herein may also refer to a plasma concentration of one or more metabolites of the active ingredient. As used herein, the term “metabolite” in relation to an active ingredient refers to a compound that is produced as a result of metabolism, degradation, oxidation, or the like, of the active ingredient. The metabolite may be produced as a result of enzyme action, oxidation processes, degradation processes and the like.

In other therapeutic applications, it is desirable to induce noticeable perceptual effects (such as hallucinogenic, mystical or spiritual effects). Such effects have been proposed to provide enhanced therapeutic effects in combination with psychotherapy. Therefore, in some embodiments, psychotherapy can be provided while the serum level of the tryptamine is above a threshold level-being a level whereby there are noticeable perceptual effects, such as hallucinations. Accordingly, in some embodiments of the methods of the invention, the composition includes a dose of the tryptamine at or above a dose required to induce noticeable perceptual effects, such as hallucinations.

Also provided is a method of enhancing the effect of a tryptamine in a subject including the steps of administering a single dosage form pharmaceutical or a composition comprising a monoamine oxidase inhibitor (MAOI) and a tryptamine, wherein the tryptamine is a substrate for a MAO. In some embodiments, the tryptamine is a psychedelic tryptamine.

In some embodiments, the effect of the tryptamine is a hallucinogenic effect. In some embodiments, the effect of the tryptamine is a therapeutic effect.

Compositions of this invention for oral administration include both solid and liquid dosage forms. Solid dosage forms for oral administration include capsules, tablets, pills, powders, films, gels and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, troches, nanoparticles and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract. Examples of embedding compositions which can be used include polymeric substances and waxes. The active compounds can also be in microencapsulated form (such as microbeads) if appropriate, with one or more of the above-mentioned excipients.

Liquid dosage forms for oral administration include pharmaceutically acceptable emulsions, solutions, gels, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof. Suspensions for administration may also include forms such as powders, nanoparticles and the like.

The invention, in other embodiments, provides a pack or kit comprising one or more containers filled with one or more of the ingredients of the compositions of the invention. In such a pack or kit can be found a container having a unit dosage of the agent(s). The kits can include a composition comprising an effective agent either as concentrates (including lyophilized compositions), which can be diluted further prior to use or they can be provided at the concentration of use, where the vials may include one or more dosages. Conveniently, in the kits, single dosages can be provided in sterile vials so that vials may be employed directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

As disclosed herein, the present invention relates to compositions comprising a monoamine oxidase inhibitor (MAOI) that is formulated for immediate release and a tryptamine that is formulated for delayed release, wherein the tryptamine is a substrate for a MAO. In certain embodiments, the composition comprises discrete particles of the MAOI and the tryptamine formulated accordingly. In certain embodiments, the composition is a single dosage form, wherein the dosage form contains discrete particles of the MAOI and the tryptamine, each formulated accordingly. Without wishing to be bound by theory, the present inventors have found that where the composition is a single dosage form comprising both the MAOI formulated for immediate release and a tryptamine for delayed release, the material that contains each of the formulated active ingredients should be selected so as to ensure that the release properties (i.e. immediate release or delayed release) of each active ingredient is maintained. In certain embodiments, the composition is in the form of a single dosage form consisting of a MAOI formulated for immediate release and a tryptamine formulated for delayed release, wherein the tryptamine is a substrate for a MAO. Accordingly, the present invention provides a method for the treatment of a neurodevelopmental, neurological, movement, psychological or physiological, disease, disorder, syndrome or symptom in a subject, the method comprising administering a monoamine oxidase inhibitor (MAOI) and a tryptamine, derivative thereof or pharmaceutically acceptable salt thereof, wherein the MAOI is formulated for immediate release and the tryptamine is formulated for delayed release, wherein the tryptamine is a substrate for a MAO, and wherein the composition is a single dosage form. The present inventors believe that the use of a single dosage form improves patient compliance and subsequently, patient experience. This may then increase the chances of successful treatment.

In certain embodiments, the compositions disclosed herein are pharmaceutical compositions.

As used herein the terms “treating”, “treatment”, “preventing”, “prevention”, “ameliorating” and “amelioration” and grammatical equivalents refer to any and all uses which remedy the stated disease, prevent, retard or delay the establishment of the disease, or otherwise prevent, hinder, retard, or reverse the progression of the disease. Thus the terms “treating” and “preventing” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

The term “therapeutically effective amount” or “effective amount” is an amount sufficient to effect beneficial or desired clinical results. An effective amount can be administered in one or more administrations.

The term “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The methods disclosed herein include the administration of a tryptamine. Any reference to a tryptamine throughout the specification is also taken to include a pharmaceutically acceptable salt thereof, a prodrug thereof, a derivative of a tryptamine and the pharmaceutically acceptable salts thereof. As used herein, the term “derivative” in relation to a tryptamine refers to a tryptamine that includes one or more substituents attached to the tryptamine, where the substituent may be further fused (so as to form a polycyclic system) with one or more non-hydrogen groups. In certain embodiments, the derivative of the tryptamine has one or more substituents attached to the indole ring of the tryptamine. In certain embodiments, the derivative of the tryptamine has one or more substituents attached at the 4-, 5- and/or 6-positions of the indole ring of the tryptamine. In other embodiments, the derivative of the tryptamine has one or more substituents attached to the terminal amine group. In some embodiments, the derivative of the tryptamine has one or more substituents, wherein the substituents are the same or different. In some embodiments, the tryptamine is a psychedelic tryptamine.

In certain embodiments, the one or more substituents present on the derivative are independently selected from the group consisting of halogen, deutero, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, glycosyl, phosphoryl, —C(═O)OH, —C(═O)R^{a}, —C(═O)OR^{a}, C(═O)NR^{a}R^{b}, C(═NOH)R^{a}, C(═NR^{a})NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NR^{b})NR^{c}R^{d}, NR^{a}SO₂R^{b}, —SR^{a}, SO₂NR^{a}R^{b}, —OR^{a}, OC(═O)NR^{a}R^{b}, OC(═O)R^{a} and acyl, wherein R^{a}, R^{b}, R^{c} and Rd are each independently selected from the group consisting of H, C₁-C₁₂alkyl, C₁-C₁₂haloalkyl, C₂-C₁₂alkenyl, C₂-C₁₂alkynyl, C₂-C₁₀ heteroalkyl, C₃-C₁₂cycloalkyl, C₃-C₁₂cycloalkenyl, C₂-C₁₂heterocycloalkyl, C₂-C₁₂ heterocycloalkenyl, C₆-C₁₈aryl, C₁-C₁₈heteroaryl, and acyl, or any two or more of R^{a}, R^{b}, R^{c} and Rd, when taken together with the atoms to which they are attached form a heterocyclic ring system with 3 to 12 ring atoms.

In some embodiments, each substituent is independently selected from the group consisting of: halogen, deutero, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, acyl, glycosyl and phosphoryl.

Examples of particularly suitable substituents include F, Cl, Br, I, C₁-C₁₂ alkyl, OH, OCH₃, CF₃, OCF₃, NO₂, NH₂, COOH, COOCH₃, CN, glycosyl and phosphoryl. In certain embodiments, the derivative of a tryptamine contains a halo substitution on the indole core of the tryptamine. In certain embodiments, the halo substitution is selected from the group consisting of fluoro, chloro, bromo and iodo. In certain embodiments, the derivative of a tryptamine contains a 4,5-methylenedioxy group attached to the indole core of the tryptamine. In other embodiments, the derivative of a tryptamine contains a 5,6-methylenedioxy group attached to the indole core of the tryptamine. In certain embodiments, the derivative of a tryptamine contains a 4-hydroxy, 5-hydroxy or 6-hydroxy group on the indole core of the tryptamine. In other embodiments, the derivative of the tryptamine contains an N-alkyl group on the terminal amine of the tryptamine core. In certain embodiments, the derivative of a tryptamine contains one or more substituents selected from the group consisting of N-methyl, N-ethyl, N-propyl, N-isopropyl, N-butyl and N-isobutyl, wherein the one or more substituents may be the same or different. In other embodiments, the derivative of a tryptamine contains a methoxy group attached to the indole core of the tryptamine. In other embodiments, the derivative of a tryptamine contains a thioalkyl group attached to indole core of the tryptamine. In certain embodiments, the derivative of a tryptamine contains one or more acyloxy groups attached to the indole core of the tryptamine. In other embodiments, the derivative of a tryptamine contains one or more alkoxy groups attached to the indole core of the tryptamine. In some embodiments, the tryptamine on which the one or more substituents is located is a psychedelic tryptamine.

Tryptamines, having structural similarity to serotonin, can bind to serotonin receptors such as 5-HT2A, 5-HT1A or 5-HT2C, which appear to be critical for their psychotropic effects. Psychedelic tryptamines include (but are not limited to) N,N-dimethyltryptamine (DMT) and its derivatives alpha-methyltryptamine (AMT), 5-methoxy-N, N-dimethyltryptamine (5-MeO-DMT) and 5-methoxy-N,N-diisopropyltryptamine (5-MeO-DIPT), psylocibin ([3-(2-dimethylaminoethyl)-1H-indol-4-yl] dihydrogen phosphate and its metabolite psilocin (4-hydroxy-N,N-dimethyltryptamine, 4-OH-DMT). The structures of serotonin, tryptamine and various psychedelic tryptamines can be found in Figure A.

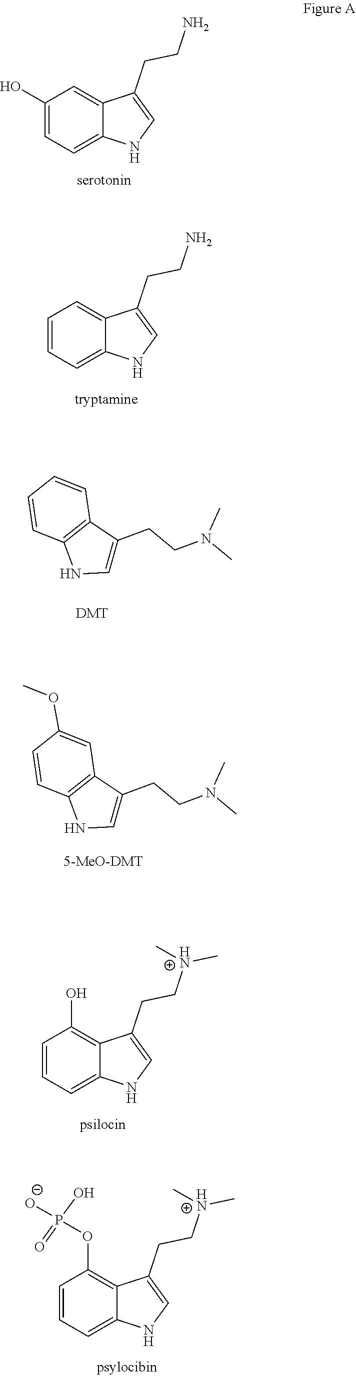

In some embodiments, the psychedelic tryptamine is selected from the group consisting of psilocybin, a prodrug of psilocin, N,N-dimethyltryptamine (DMT), 5-methoxy-N, N-dimethyltryptamine (5-MeO-DMT) and pharmaceutically acceptable salts thereof. In some embodiments, the prodrug of psilocin is psylocibin.

In some embodiments, the psychedelic tryptamine is N,N-dimethyltryptamine (DMT) or a pharmaceutically acceptable salt thereof.

In certain embodiments, the derivatives of a psychedelic tryptamine include 2-methyl-diethyltryptamine (2-Me-DET), 4-Hydroxy-N,N-dibutyltryptamine (4-HO-DBT), 4-hydroxy-diethyl-tryptamine (4-HO-DET or CZ-74), 4-Hydroxy-N,N-diisopropyltryptamine (4-HO-DiPT or Iprocin), 4-HO-EPT (4-hydroxy-N-ethyl-N-propyltryptamine), 4-HO-McPT (4-hydroxy-N-methyl-N-cyclopropyltryptamine), 4-HO-MET (4-hydroxy-N-methyl-N-ethyltryptamine, metocin or methylcybin), 4-HO-MiPT (miprocin, 4-hydroxy-N-methyl-N-isopropyltryptamine), 4-HO-MPMI (4-Hydroxy-N-methyl-(α,N-trimethylene)-tryptamine or lucigenol), 4-Hydroxy-N-methyl-N-propyltryptamine (4-HO-MPT or meprocin), 4-Hydroxy-α-methyltryptamine (4-HO-αMT), 4-methoxy-N-methyl-N-isopropyltryptamine (4-MeO-MiPT), 4-Methyl-α-methyltryptamine (4-Me-αMT), 4-Propionoxy-N,N-dimethyltryptamine (4-PrO-DMT or O-Propionylpsilocin), 4,5-methylenedioxy-N,N-diisopropyltryptamine (4,5-MDO-DiPT), 4,5-methylenedioxy-N,N-dimethyltryptamine (4,5-MDO-DMT), 5-Ethoxy-alpha-methyltryptamine (5-EtO-AMT), 5-Fluoro-α-ethyltryptamine (5-F-AET), 5-fluoro-N, N-diethyltryptamine (5F-DET or 5-Fluoro-DET), 5-Fluoro-N,N-dimethyltryptamine (5-fluoro-DMT or 5F-DMT), 5-fluoro-N-ethyl-N-propyltryptamine (5-Fluoro-EPT or 5F-EPT), 5-fluoro-N-methyl-N-ethyltryptamine (5-Fluoro-MET or 5F-MET), 5-Methoxy-alpha-ethyltryptamine (5-MeO-α-ET), 5-methoxy-α-methyltryptamine (5-MeO-αMT), a, O-Dimethylserotonin (Alpha-O), N,N-diallyl-5-methoxytryptamine (5-MeO-DALT), 5-methoxy-N,N-diethyltryptamine (5-MeO-DET), 5-methoxy-N,N-Dipropyltryptamine (5-MeO-DPT), 5-methoxy-N-ethyl-N-Isopropyltryptamine (5-MeO EiPT), 5-methoxy-N-methyl-N-allyltryptamine (5-MeO-MALT), 5-Methoxy-N,N-Methylisopropyltryptamine (5-MeO-MiPT), 5-Methoxy-N-methyl-(α, N-trimethylene) tryptamine (5-MeO-MPMI), 5-methoxy-N,N-tetramethylenetryptamine (5-MeO-pyr-T), 5-methylthio-N,N-dimethyltryptamine (5-MeS-DMT), 5-Methoxy-2,N,N-trimethyltryptamine (5-MeO-2,N,N-TMT or 5-MeO-TMT), 5-(trifluoromethyl)-N,N-dimethyltryptamine (5-TFM-DMT, (5-(trifluoromethyl)-DMT or 5-CF3-DMT), 5-(trifluoromethoxy)-N,N-dimethyltryptamine (5-TFMO-DMT, 5-(trifluoromethoxy)-DMT or 5-OCF3-DMT), 5,6-methylenedioxy-N,N-diisopropyltryptamine (5,6-MDO-DiPT), 5,6-methylenedioxy-N,N-dimethyltryptamine (5,6-MDO-DMT), 5,6-methylenedioxy-N-methyl-N-isopropyltryptamine (5,6-MDO-MiPT), 5,6-dimethoxy-N-methyl-N-isopropyltryptamine (5,6-MeO-MiPT), 5,N, N-trimethyltryptamine (5,N,N-TMT or 5-TMT), 5-fluoro-3-[(2R)-1-methylpyrrolidin-2-yl]methyl]-1H-indole (5F-MPMI), 6-Fluoro-DET (6F-DET or 6-fluoro-N,N-diethyltryptamine), 6-Fluoro-N,N-dimethyltryptamine (6-Fluoro-DMT), 6-methoxy-1,2,3,4-tetrahydroharman (6-MeO-THH), 7-Chloro-α-methyltryptamine (7-CI-AMT), 7,N,N-trimethyltryptamine (7-methyl-DMT or 7-TMT), 7-Methyl-α-ethyltryptamine (7-Me-αET), 5-Methoxy-7-fluoro-N-methyl-N-ethyltryptamine (7-F-5-MeO-MET, 5-MeO-7-F-MET), 1-isopropyl-6-fluoro-psilocin (O-4310), 5-Methoxy-7-fluoro-N-methyl-N-ethyltryptamine (7-F-5-MeO-MET or 5-MeO-7-F-MET), 4-Acetoxy-N,N-diethyltryptamine (4-Acetoxy-DET, ethacetin, ethylacybin or 4-AcO-DET), 4-acetoxy-N,N-diisopropyltryptamine (4-Acetoxy-DiPT or ipracetin), 4-Acetoxy-N-methyl-N-ethyltryptamine (4-Acetoxy-MET), 4-acetoxy-N-methyl-N-isopropyltryptamine (4-AcO-MiPT or mipracetin), O-Acetylbufotenine (5-AcO-DMT or bufotenine acetate), N,N,N-trimethyl-4-phosphoryloxytryptamine (O-Acetylpsilocin, psilacetin, 4-acetoxy-DMT, 4-AcO-DMT or Aeruginascin), α,N-dimethyltryptamine (Alpha,N-DMT), α,N-dimethyl-5-methoxytryptamine (Alpha,N,O-TMS), Baeocystin, 4-hydroxy-N-methyltryptamine (4-HO-NMT), 5-bromo-N,N-dimethyltryptamine (5-Bromo-DMT), Bufotenin (5-HO-DMT, bufotenine), 5-Chloro-α-methyltryptamine (5-Chloro-αMT or PAL-542), N,N-Diallyltryptamine (DALT), N,N-Dibutyltryptamine (DBT), N,N-diethyltryptamine (DET or T-9), Diisopropyltryptamine, N, N-diisopropyltryptamine (DiPT), 5,N-Dimethyl-N-isopropyltryptamine (5-Me-MiPT), N,N-Dipropyltryptamine (DPT), α-Dimethyltryptamine (2,α-DMT), Ethocybin (CEY-19, 4-phosphoryloxy-DET or 4-PO-DET), Ethylisopropyltryptamine (EiPT), α-Ethyltryptamine (αET, AET or etryptamine), 5-Fluoro-α-methyltryptamine (5-Fluoro-αMT, 5F-AMT or PAL-544), 4-Fluoro-5-Methoxy-N,N-dimethyltryptamine (4-F-5-MeO-DMT), 4-glutaryloxy-N,N-diisopropyltryptamine (FT-104), 5-Methoxy-N,N-diisopropyltryptamine (5-MeO-DiPT, foxy methoxy or foxy), 4-Methyl-α-ethyltryptamine (4-Me-αET), N-Methyl-N-ethyltryptamine (MET), N-methyl-N-butyltryptamine (MBT), N-Methyl-N-isopropyltryptamine (MiPT), α-methyl-5-hydroxytryptamine or 5-hydroxy-α-methyltryptamine (α-methyl-5-HT, 5-HO-αMT, α-Methylserotonin or αMS), α-Methyltryptamine (αMT, AMT), N-Methyltryptamine (NMT), 3-(N-methylpyrrolidin-3-ylmethyl) indole (MPMI), Norbaeocystin, Norpsilocin, Propylisopropyltryptamine (PiPT), N,N-tetramethylenetryptamine (Pyr-T), 2,N,N-trimethyltryptamine (2,N,N-TMT or 2-Me-DMT), and α,N,N-Trimethyltryptamine (α,N,N-TMT, α-TMT or ATMT).

As used herein, the term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic acids. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids:acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

Known salts of DMT include the fumarate, citrate, acetate, bioxalate and hydrochloride salts. Further, freebase forms of DMT are known in the art.

“N,N-dimethyltryptamine” or “DMT” (also known as dimethyltryptamine, 2-(3-indolyl)ethyldimethylamine or 3-(2-dimethylaminoethyl) indole) refers to a psychedelic compound found in many plant species including (but not limited to) Acacia species, Anadenanthera species (including Anadenanthera peregrina and Anadenanthera colubrina), Desmanthus illinoensis, Diplopterys cabrerana, Mimosa species (including Mimosa tenuiflora and Mimosa pudica), Psychotria viridis, and Virola species.

Deuterated versions of N,N-dimethyltryptamine such as α,α-dideutero-N,N-dimethyltryptamine, α,α,β,β-dideutero-N,N-dimethyltryptamine, α-protio, α-deutero-N,N-dimethyltryptamine compounds are known in the art. In these forms, the alteration of the alpha and beta hydrogens on the side chain of DMT (and its structural relative such as 5-MeO-DMT-see below) may protect the molecule from MAO metabolism and prolong its in vivo half-life and effects. Such modification can also alter the required dosage, with deuterated versions resulting in 2-3 times greater concentration of DMT in the brain compared to the same dose of DMT.

The hallucinogenic effects of DMT in the form of ayahuasca (typical dosage of 0.6-4 mg/kg DMT) generally appear within 60 min, peak at 90 min and subside at up to 4 hours. Typical doses of smoked or inhaled free-base DMT are in the range of 40-50 mg, and up to 100 mg.

Other dosage routes of free-base DMT such as inhaled DMT (0.07-0.28 mg/kg) and rectally administered DMT (1.7 mg/kg of the bioxalate salt) have not shown effectiveness. Accordingly, the current effective means for consumption of DMT are intravenous or intramuscular injection, or smoking. However, such means of delivery are not practical for use in a clinical setting. Alternatively, oral consumption of DMT is effective when administered in the form of liquid ayahuasca comprising harmaline (or harmala alkaloids) and DMT as active ingredients.

In some embodiments, the dimethyltryptamine is 5-methoxy-N,N-dimethyltryptamine (5-MeO-DMT). As used herein, “5-MeO-DMT” refers to a psychedelic compound found in the plant Dictyoloma incanescens and the venom of the Sonoran Desert Toad (Bufo alvarius/Incilius alvarius). 5-MeO-DMT is also known as N, N-Dimethyl-5-methoxytryptamine, 5-Methoxy-N,N-dimethyltryptamine, Methoxybufotenin and O-Methylbufotenine.

Other mono-N-methyltryptamines with psychotropic effects are known in the art including: 5-MeO-DMT, 5-MeS-DMT, 5-HO-DMT (bufoteneine), 5-MeO-DPT (5-methoxy-N,N-dipropyltryptamine), AMT (2-(1H-indol-3-yl)-1-methyl-ethylamine), 4-AcO-DMT (4-acetoxy-N,N-dimethyltryptamine), 4-AcODiPT (4-Acetoxy-N,N-diisopropyltryptamine)N-ethyl-N-propyl-tryptamine (EPT) and N-methyl-N-allyl-tryptamine (MALT).

5-MeO-DMT has approximately 5-6 times the potency of DMT. However, it is reported to produce reduced auditory hallucinogenic effects. Exogenously administered 5-MeO-DMT is a potent, short-lived and quick-acting hallucinogen. The concentration required to induce hallucinations is dependent on the route of administration. For example, inhalation requires ˜6-20 mg, intravenous injection requires ˜0.7-3.1 mg, sublingual or intranasal insufflation requires ˜10 mg, and oral administration requires ˜30 mg. Notably, the plasma concentration at which an individual experiences noticeable perceptual effects (such as hallucinations) may vary from individual to individual, and therefore may need to be determined on an individual basis. Alternatively, a threshold serum concentration, above which there are noticeable perceptual effects, can be based on the observed, calculated or estimated threshold of a population, such as the median or mean threshold observed in a comparative population.

In a mouse model, oral administration of various concentrations of 5-MeO-DMT (2 mg/kg and 10 mg/kg) subsequent to (15 minutes after) administration of various concentrations of Harmaline resulted in the plasma concentrations as disclosed in Table 1 of Jiang X L et al. Drug Metabolism and Disposition 2013; 41 (5): 975-86.

As used herein, “psilocin” (also psilocin or psilocine) refers to the psychedelic compound 3-[2-(dimethylamino)ethyl]-1H-indol-4-ol as found in certain mushroom genera, such as Copelandia, Gymnopilus, Inocybe, Panaeolus, Pholiotina, Pluteus, and Psilocybe (or synthesized and biosynthesized forms of the compound). Psilocin is also known by the synonyms Psilocine, 4-Hydroxy-N,N-dimethyltryptamine or Psilotsin.

As used herein, “psilocybin” refers to the psychedelic compound [3-[2-(dimethylamino)ethyl]-1H-indol-4-yl] dihydrogen phosphate as found in certain mushroom species, such as those in the genus Psilocybe. Psylocibin is also known by the synonyms Psilocibin, Psilocybin and Psilocybine. Psylocibin is a phosphorylated form of psilocin, having a phosphate group in place of the hydroxyl group at position 4 of psilocin. Psylocibin is rapidly dephosphorylated by alkaline phosphatases (and other non-specific esterases) in the liver and converted into psilocin. Alternatively, when orally administered, psylocibin can be dephosphorylated in the acid environment of the stomach. As a result, psylocibin is a prodrug of psilocin. Psilocin is about 1.4 times as potent as psilocybin on a weight-to-weight basis, which corresponds to the relative molecular weight of each substance.

As used herein, the term “prodrug” refers to and includes derivatives that are converted in vivo to the compounds of the present invention. Such derivatives would readily occur to those skilled in the art, and include, for example, compounds containing a free hydroxyl group that is converted into an ester derivative, or containing a ring nitrogen atom that is converted to an N-oxide. Examples of ester derivatives include alkyl esters, phosphate esters and those formed from amino acids.

The present invention relates to methods of treatment that include the step of administering a tryptamine and compositions comprising a tryptamine. In certain embodiments, the tryptamine is a psychedelic tryptamine. Psychedelic tryptamines, such as DMT, psilocin and 5-MeO-DMT, are monoamines and as such are metabolised, at least in part, by peripheral monoamine oxidases (MAO). This means that oral consumption of many psychedelic tryptamines results in poor bioavailability due to extensive first pass metabolism. However, even once within the systemic circulation, ongoing metabolism of psychedelic tryptamines by MAOs can result in rapid clearance.

Such rapid metabolism can be seen in several naturally derived psychedelic tryptamines such as DMT and psilocin. As seen in Figure B, monoamine oxidase A converts DMT to indole-3-acetic acid (IAA). Analysis of post administration blood samples and urine after oral administration of DMT showed no unmetabolized DMT. However, 97% of recovered compounds were MAO-dependent IAA, whereas cytochrome P450 dependent DMT-N-oxide (DMT-NO) accounted for only 3%. If first-pass metabolism was circumvented by smoking DMT and thus avoiding gastro-intestinal tract absorption, only 63% of recovered metabolites were the MAO-dependent IAA. Notably, psychotropic effects were not experienced after oral consumption of DMT—but were noted after smoking an equivalent dose.

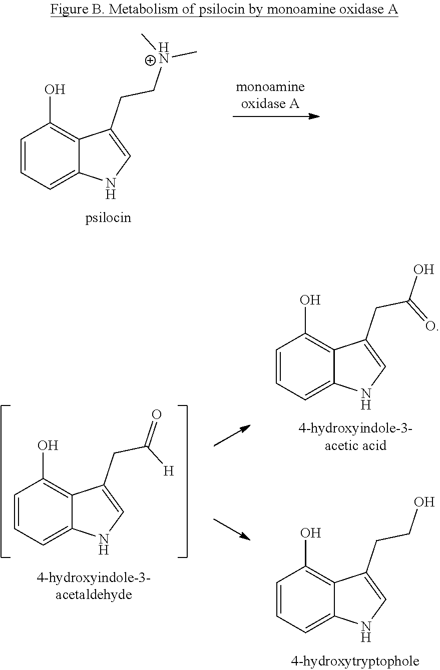

Similarly, psilocin (the pharmacologically active form of psylocibin) is also metabolised by MAO to an intermediate (4-hydroxy-indole-3-acetaldehyde) and ultimately 4-hydroxy-indole-3-acetic acid and 4-hydroxy-indole-3-acetaldehyde.

In all of these reactions, the tryptamine is deamidated and oxidised at the amine of the aminoethyl group. Accordingly, in some embodiments, the tryptamine includes an aminoethyl group.

Because tryptamines can be enzymatically metabolised by MAO the pharmacokinetics can be altered by mono-amine oxidase inhibition, for example by using mono-amine oxidase inhibitors (MAOIs). However, inhibition of MAO is often delayed (30 minutes to 2 hours) after ingestion or administration of a MAOI. However, most psychedelic tryptamines are rapidly absorbed and fast acting. As a result of this, concurrent consumption or administration of a psychedelic tryptamine and an MAOI is generally ineffective because the tryptamine is metabolised before the MAO is inhibited. Without wishing to be bound by theory, the present inventors believe that in order to alter the pharmacokinetics of the tryptamine, the MAOI should be administered in advance of the tryptamine.

Therefore in another aspect, the present invention provides a method of treating a neurodevelopmental, neurological, movement, psychological or physiological, disease, disorder, syndrome or symptom in a subject, the method comprising administering a composition comprising a monoamine oxidase inhibitor (MAOI) and a tryptamine, wherein the MAOI is formulated for immediate release and the psychedelic tryptamine is formulated for delayed release, and wherein the tryptamine is a substrate for a MAO. In certain embodiments, the tryptamine is a psychedelic tryptamine.

In certain embodiments, the psychedelic tryptamine is in the form of a modified-release. Dosage forms can be designed to modify the release of the drug over a given time or after the dosage form reaches the required location.

In some embodiments, the modified-release is a delayed release, sustained-release or extended-release.

As used herein, the term “release” in the context of an active ingredient that is formulated in a dosage form for “immediate release” or “delayed release”, for example, refers to the dissolution of the dosage form and diffusion of the active ingredient into the medium into which the dosage form is delivered. The release of an active ingredient from its dosage form is typically governed by various parameters such as the physicochemical properties of the active itself, the physical and/or chemical characteristics of the matrix in which the active ingredient is located, the pH and temperature of the release environment, for example.

As used herein, the term “delayed release” refers to a dosage form that is formulated to release the active ingredient at a time other than immediately after administration of the dosage form to the subject. A delayed release oral dosage form may allow for control of where the drug is released, for example, when the dosage form reaches the small intestine after passing the stomach. For example, a delayed release dosage form may remain intact upon administration to the subject and dissolve to release the active ingredient after a time of 1 hour, 2 hours, 3 hours, 4, hours, 5 hours, 6 hours and the like. A delayed release form of the composition may also extend the time period during which the active ingredient is released, such that the dosage form is also an extended release or sustained release dosage form where the active ingredient is released over a number of minutes, hours or the like. Extended-release systems allow for the active ingredient to be released over prolonged time periods. By extending the release profile of an active ingredient, the frequency of dosing can be reduced or the time above a therapeutic threshold can be increased. Extended release can be achieved using sustained- or controlled-release dosage forms.

A range of systems and formulations are known in the art to delay, extend or control the release of pharmaceutical agents. These include, but are not limited to diffusion systems, dissolution systems, osmotic controlled release oral delivery systems, ion-exchange resins, floating systems, matrix systems and stimuli inducing systems. Often polymers are used to achieve delayed release in diffusion systems. The dosage form (for example, a tablet or the granules before tableting) can be coated with a suitable polymer. The polymer dissolves as a function of pH. This means that release of the polymer-coated active agent can be targeted for the low-pH environment of the stomach or the higher-pH environment of the small intestine. Upon dissolution of the polymer coating, the release of the active agent can be immediate and the resulting plasma concentration versus time curve is similar to the one for immediate-release dosage forms, with a suitable off set.

Accordingly, in some embodiments, the composition comprises at least one coating (e.g. at least partially, or wholly) encapsulating or containing at least a portion of the psychedelic tryptamine. In certain embodiments, the controlled release coating is a stable controlled release monolithic coating that is formed by a process that comprises coating the psychedelic tryptamine with a coating composition.

In some embodiments, the polymer coating hydrates when placed into water. In some embodiments, the dosage form floats in water. In some embodiments, the (e.g. controlled release) dosage form, upon oral administration to an individual, provides controlled release of an effective amount of the active ingredient to at least one region of the patient's upper gastrointestinal tract (e.g. the stomach).

In certain embodiments of the present invention comprising a coated form of an active ingredient, the length and time for the delay of the release of the active ingredient may be controlled by rate of hydration and the thickness of the coat. The drug release rate subsequent to the delay may in part, be determined by the thickness and permeability of the hydrated coat. Thus, it is possible to regulate the rate of hydration and permeability of the coat so that the desired controlled release drug profile might be achieved. Other parameters in combination with the thickness of the coat include varying the concentrations of some of the ingredients of the stable coat composition and/or varying the curing temperature and length of curing the coated tablet cores. The person skilled in the art will know which parameters or combination of parameters to change for a desired controlled release profile.

In some embodiments, the composition will comprise an extended-release component. In certain embodiments, the extended-release component comprises the tryptamine.

In some embodiments, the composition comprises a sustained release component. These sustained-release systems maintain the rate of drug release over a sustained period. For example, if the release of the drug from the dosage form is sustained such that the release takes place throughout the entire gastrointestinal tract, one could reduce Cₘₐₓ and prolong the time interval of drug concentration in the therapeutic range. Sustained-release dosage forms achieve this mostly by the use of suitable polymers, which are used either to coat granules or tablets (reservoir systems) or to form a matrix in which the drug is dissolved or dispersed (matrix systems). In certain embodiments, the sustained-release component comprises a tryptamine. In some embodiments, the sustained-release component comprises a psychedelic tryptamine.

In some embodiments, the composition comprises a controlled-release component. Controlled-release systems also offer a sustained-release profile but, in contrast to sustained-release forms, controlled-release systems are designed to lead to predictably constant plasma concentrations, independently of the biological environment of the application site. This means that the concentration of the drug in the body is also controlled, not just the release of the drug from the dosage form, as is the case in a sustained-release system. In certain embodiments, the controlled-release component comprises a tryptamine.

Without wishing to be bound by theory, the present inventors believe that the extended-release compositions disclosed herein may also have other release properties, for example, sustained-release and/or controlled-release. This means that a composition comprising a specific active ingredient may have both delayed-release and extended-release properties. In certain embodiments of the present invention, the compositions disclosed herein comprise a tryptamine that is formulated for both delayed-release and extended-release.

The present invention also relates to methods of treatment that include the step of administering a monoamine oxidase inhibitor (MAOI) and compositions comprising a MAOI. In certain embodiments, the present invention provides methods that include the administration of a monoamine oxidase inhibitor (MAOI), where the MAOI is formulated for immediate release. In certain embodiments, the MAOI is a reversible MAOI.

As used herein, the term “immediate release” refers to a dosage form that is formulated to substantially release (e.g. release at least or about 80%, 85%, 90%, 91%, 92%, 93%, 94%, 95%, 96%, 97%, 95% or 99%) the active ingredient without delay (e.g. within about or no more than 2 minutes, 5 minutes, 10 minutes, 15 minutes, 20 minutes, 25 minutes, 30 minutes, 35 minutes, 40 minutes, 45 minutes, 50 minutes, 55 minutes or 60 minutes) upon administration of the dosage form to the subject. Where the dosage form includes a coating that surrounds the active ingredient, the coating disintegrates rapidly and does not persist, thus allowing the active ingredient to diffuse and subsequently be incorporated into the subject. Immediate-release dosage forms usually release (dissolve or disperse) the drug in a single action following a first-order kinetics profile. This means the drug is released initially very quickly and then passes through the mucosal membrane into the body, reaching the highest plasma level (termed Cₘₐₓ) in a comparatively short time (termed tₘₐₓ). Uptake through the mucosal membranes may be due to passive diffusion or by receptor-mediated active transport mechanisms (see section on modified release). Once taken up into the body the drug is distributed throughout the body and elimination of the drug by metabolism and excretion occurs. The elimination process also usually follows first-order kinetics. Therefore, the plasma levels measured over time after administration of an immediate-release dosage form (i.e. the plasma concentration-time curve) resembles the sum of a first-order absorption and a first-order elimination process. The resulting function is known as the Bateman function. For example, immediate release of an active ingredient includes but not limited to contact with saliva or gastric juices and results in substantially complete dissolution within (for example) about 2 minutes, 5 minutes, 10 minutes, 20 minutes or 30 minutes). Immediate-release components might also be referred to as instant release. When used in association with the dissolution profiles discussed herein, the term “immediate-release” refers to that portion of a composition as disclosed herein which delivers active agent over a period of time, such as those described herein in relation to MAOI.

As used herein, the term “monoamine oxidase inhibitor” (MAOI) refers to a class of pharmacologically active compounds that inhibit the activity of one or more monoamine oxidase (MOA) enzymes, i.e. monoamine oxidase A (MAO-A) or monoamine oxidase B (MAO-B). In some embodiments, the MOAI is a reversible inhibitor of MOA. In other embodiments, the MAOI is a non-reversible inhibitor of MOA. In some embodiments, the MAOI is selective for MAO-A. In other embodiments, the MAOI is selective for MAO-B. In other embodiments, the MAOI is a non-selective inhibitor. In certain embodiments, the MAOI is a reversible, non-selective inhibitor of MOA. In other embodiments, the MAOI is a non-reversible, non-selective inhibitor of MOA. In yet other embodiments, the MAOI is a reversible, selective inhibitor of MOA. In other embodiments, the MAOI is a non-reversible selective inhibitor of MOA. In some embodiments, the MAOI is a reversible, selective inhibitor for MAO-A.

Monoamine oxidase inhibitors are known in the art, and any such suitable MAOI can be used in the present invention. For example the MAOI can be selected from the group consisting: of: 1,2,3,4-tetrahydro-ß-carboline, 1,2,3,4-tetrahydroisoquinoline (TIQ), 2-methyl-1,2,3,4-tetrahydro-ß-carboline, Befloxatone, Benmoxin, Bifemelane, Brofaromine, Caroxazone, Clorgyline, Curcumin, Eprobemide, Harmaline, Harmane,

Harmine, Hydracarbazine, Iproclozide, Iproniazid, Isocarboxazid, Mebanazine, Methylene blue, Methylthioninium chloride, Metralindole, Minaprine, Moclobemide, Nialamide, Norharmane, Octamoxin, Pargyline, Phenelzine, Pheniprazine, Phenoxypropazine, Pirlindole, Pivalylbenzhydrazine, Procabazine, Rasagiline, Rosiridin, Safinamide, Safrazine, Selegiline, Tetrahydroharmine, Toloxatone, and Tranylcypromine. Preferred MAOIs for use in the present invention are those currently approved for use by a regulatory body. These include Bifemelane, CX157, Hydracarbazine, Iproniazid, Isocarboxazid, Methylthioninium chloride, Moclobemide, Phenelzine, Pirlindole, Tranylcypromine, Rasagiline, Selegiline, and Safinamide.

In some embodiments, the MOAI is a reversible inhibitor of MOA (RIMA). In certain embodiments, the reversible MAOI is selected from the group consisting of Brofaramine, Befloxatone, CX157, Moclobemide and Toloxatone. In other embodiments, the MAOI is a selective inhibitor selected from inhibitors of MAO-A, Brofaromine, Metralindole, Minaprine, Moclobemide, Pirlindole and Toloxatone.

In some embodiments, the MAOI is specific for MAO-A and reversible upon administration and binding NAOI. In certain embodiments, to the most preferred MAOI is Moclobemide due to its selective and reversible inhibition of MAO-A.

“Moclobemide” (4-chloro-N-(2-morpholin-4-ylethyl)benzamide, Figure C) is a reversible monoamine oxidase A inhibitor is also known as Aurorix, Moclobemid and Manerix.

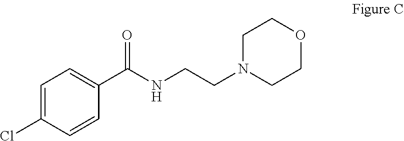 [SMILES: O=C(NCCN1CCOCC1)c1ccc(Cl)cc1]

As discussed above, the present invention provides a composition comprising a monoamine oxidase inhibitor (MAOI) and a tryptamine, wherein the MAOI is formulated for immediate release and the tryptamine is formulated for delayed release, wherein the tryptamine is a substrate for a MAO.

In certain embodiments, the composition comprising a MAOI formulated for immediate release and a tryptamine formulated for delayed release is provided as a single dosage form. In certain embodiments, the composition comprises discrete components of the MAOI formulated for immediate release and the tryptamine formulated for delayed release.

In certain embodiments, the composition comprising a MAOI formulated for immediate release and a tryptamine formulated for delayed release is provided as a single dosage form.

As used herein, the term “single dosage form” refers to a dosage form that contains a MAOI and a tryptamine in a single unit. The single dosage form may be of any suitable construction, for example, a capsule comprising discrete particles of the MAOI and the tryptamine, a multilayer tablet with a first layer comprising an immediate-release MAOI and a second layer comprising a modified-release tryptamine. In some alternative embodiments, the single dosage form is a tablet comprising an immediate-release MAOI outer layer and a modified-release tryptamine inner core. In some embodiments, the single dosage form is formulated, structured or otherwise prepared for sequential release of the MAOI followed by the tryptamine.

The compositions disclosed may be in one or more different forms. The form of the composition will dictate the method of administration. Conversely, where a specific method of administration is desired, one or more forms of the composition may be indicated. Where oral administration is desired, dosage forms may include solid forms, such as pills, tablets, capsules, orally disintegrating tablets, lozenges, thin films, powders or pastes. Other oral administration forms include liquids, such as syrups, emulsions, tinctures, solutions and elixirs, and semi-liquids, such as gels. Administration by inhalation may require a composition in the form of an aerosol, a powder for inhalation, a solution to be administered by nebulizer or vaporizer or a nasal spray. Where topical administration is desired, dosage forms include creams, ointments, liniments, balms, hydrogels, gels, transdermal patches or powders. Other dosage forms that are envisaged by the present invention include parenteral compositions in the form of a solution, such as those for intradermal, subcutaneous, intramuscular, intraosseous, intraperitoneal or intravenous administration. In some embodiments, the compositions of the present invention are provided as a solid dosage form.

In certain embodiments, the composition comprising a MAOI formulated for immediate release and a tryptamine formulated for delayed release is in the form of a capsule. In other embodiments, the composition is in the form of a tablet. In other embodiments, the composition is in the form of a gel. In other embodiments, the composition is provided as a nasal gel. In other embodiments, the composition is in the form of a nasal spray. In other embodiments, the composition is in the form of a powder. In other embodiments, the composition is provided as a dry powder for inhalation. In other embodiments, the composition is provided as a thin film. In other embodiments, the composition is provided as an orally disintegrating tablet.

In some embodiments, the composition is an enteral composition or is for use as an enteral composition. In some alternative embodiments, the composition is a sublingual or buccal composition. The sublingual or buccal composition can take any form, but in some embodiments, the composition is in the form of a tablet or a film. The physical properties of sublingual formulations (e.g. hardness, friability, in vitro disintegration, in vitro dissolution, etc) and the like fall within the guidelines provided in the United States Pharmacopeia (USP).

In other embodiments, the composition is a nootropic, a supplement drink or a supplement food product. In some embodiments, the nootropic, supplement drink or supplement food is a functional food or drink.

As disclosed herein, the compositions of the present invention comprise a MAOI formulated for immediate release and a tryptamine formulated for delayed release, wherein the tryptamine is a substrate for a MAO.

In a further aspect of the present invention, there is provided a composition comprising a monoamine oxidase inhibitor (MAOI) and one or more excipients, wherein the MAOI is formulated for immediate release. In certain embodiments, the MAOI is present in an amount of between about 0.1% to about 90% by weight. In certain embodiments, the MAOI is present in an amount of about 1%, 5%, 10%, 15%, 20%, 25%, 30%, 35%, 40%, 50%, 55%, 60%, 65%, 70%, 75%, 80%, 85% or 90% in the composition.

The present inventors have found that the immediate release properties of a composition comprising a MAOI may be provided by mixing one or more disintegrants with a sufficient amount of water and the MAOI to provide a mixture that can be extruded, moulded or otherwise formed into the desired physical dosage form. In certain embodiments, the composition comprising a MAOI with immediate release properties comprises one or more disintegrants and one or more superdisintegrants. In further embodiments, the composition comprising a MAOI with immediate release properties comprises one or more disintegrants, one or more superdisintegrants and one or more sugars.

In certain embodiments, the composition comprises one or more disintegrants. In certain embodiments, the composition comprises two or more disintegrants. In certain embodiments, the one or more of the disintegrants present in the composition are selected from the group consisting of cellulose polymers, a starches and alginic acids. In certain embodiments, the composition comprises a cellulose polymer. In other embodiments, the composition comprises a microcrystalline cellulose as a disintegrant. In another embodiment, the composition comprises starch as a disintegrant.

In some embodiments, the composition comprises microcrystalline cellulose as a disintegrant. In some embodiments, microcrystalline cellulose is present in an amount of between about 0.1% to about 99% by weight of the composition. In certain embodiments, microcrystalline cellulose is present in an amount of about 1%, 5%, 10%, 15%, 20%, 25%, 30%, 35%, 40%, 50%, 55%, 60%, 65%, 70%, 75%, 80%, 85% or 90% in the composition.

The compositions of the present invention comprising a MAOI formulated for immediate release also comprise one or more superdisintegrants. In certain embodiments, the superdisintegrant is selected from the group consisting of crosslinked sodium carboxymethyl cellulose, crosslinked alginic acid, sodium starch glycolate, modified guar gum and modified agar. In some embodiments, the composition comprises a crosslinked sodium carboxymethylcellulose.

In some embodiments, the composition comprises a crosslinked sodium carboxymethylcellulose as a superdisintegrant. In some embodiments, a crosslinked sodium carboxymethylcellulose is present in an amount of between about 0.1% to about 20% by weight of the composition. In certain embodiments, a crosslinked sodium carboxymethylcellulose is present in an amount of about 0.1%, 0.5%, 1%, 2%, 4%, 6%, 8%, 10%, 12%, 14%, 16%, 18% or 20% in the composition.

As disclosed herein, the compositions comprising a MAOI formulated for immediate release also comprise one or more sugars. In certain embodiments, the one or more sugars present in the compositions disclosed herein are selected from the group consisting of lactose, mannitol, dextrose, sorbitol, maltitol, sucrose, isomalt, xylitol and glucose. In certain embodiments, the composition comprises lactose. In other embodiments, the composition comprises mannitol.

In some embodiments, the composition comprises lactose. In some embodiments, lactose is present in an amount of between about 0.1% to about 99% by weight of the composition. In certain embodiments, lactose is present in an amount of about 1%, 5%, 10%, 15%, 20%, 25%, 30%, 35%, 40%, 50%, 55%, 60%, 65%, 70%, 75%, 80%, 85% or 90% in the composition.

In certain embodiments, the composition comprising a MAOI formulated for immediate release comprises a disintegrant, a superdisintegrant, a sugar and water. In certain embodiments, the disintegrant is a microcrystalline cellulose. In some embodiments, the superdisintegrant is sodium carboxymethyl cellulose. In other embodiments, the sugar is lactose. In certain embodiments, the composition comprising a MAOI formulated for immediate release comprises microcrystalline cellulose, sodium carboxymethyl cellulose, lactose and water. In some embodiments, the MAOI present in the formulation is Moclobemide. The present inventors believe that the specific combination of a disintegrant, a superdisintegrant and a sugar provides a stable composition of the MAOI and that the stability of the composition and the release profile of the composition results from the combination of each of these components in certain amounts. One skilled in the art would understand that the amount of water required in the composition will depend on the nature of the excipients, the moisture level of each component and any physical requirements of the composition.

The present invention also provides for the preparation of a composition comprising a MAOI formulated for immediate release. The preparation of a composition comprising a MAOI as disclosed herein includes the mixing of a disintegrant, a superdisintegrant and a sugar with the MAOI and an amount of water in order to provide a mixture that is suitable for further processing into the desired dosage form. In certain embodiments, the preparation of the composition comprising MAOI includes sieving of the mixture in order to provide particles of a particular size. In other embodiments, the preparation of the composition requires the extrusion of the mixture to provide a solid form that may be further divided. The preparation of a composition comprising the MAOI may also include one or more drying steps. The present inventors believe that the combination of the excipients and the MAOI with the preparation steps as discussed herein result in a dosage form that provides sufficient stability for storage and the desired release properties. In certain embodiments, the MAOI that is formulated for immediate release is moclobemide.

As indicated above, in some forms of the invention the MAOI is formulated for immediate release. Accordingly, this may result in embodiments of the invention which are formulated for complete release of the MAOI within 2 minutes, within 5 minutes, within 10 minutes, within 15 minutes, within 20 minutes, within 25 minutes, or within 30 minutes after administration to a subject. In certain embodiments, complete release of the MAOI occurs within about 1 hour after administration to a subject. In certain embodiments, the MAOI is moclobemide. Peak plasma levels of moclobemide occur about 20 minutes to 2 hours after administration with a half-life of about 2 hours. Furthermore, inhibition of MAO by moclobemide persists for about 8 to 10 hours and is completely absent by 24 hours after administration.

The dose of the tryptamine that is delivered to the subject upon administration of a composition as described herein can be measured in accordance by more than one parameter. For example, the dose of the tryptamine may refer to the amount of the tryptamine (such as, in milligrams) that is provided in a composition and subsequently administered to a subject. The dose of the tryptamine may also take into account the weight of the subject, i.e. in mg/kg. As the physiological effect of the tryptamine requires a particular threshold to be reached, the amount of the tryptamine administered in order to reach a given concentration in plasma of the subject may also be used. Alternatively, the amount of the tryptamine that is administered to a subject may be calculated so as to ensure that the plasma concentration of the tryptamine in the subject remains below a particular threshold. Other means for describing a dose of the tryptamine that is administered may be with reference to the time over which the concentration of the tryptamine in plasma remains above (or below) a given threshold, or the time required in order to reach a peak in the plasma concentration of the tryptamine. In relation to threshold concentrations, it will be understood that the concentration for particular states, e.g. with or without noticeable perceptual effects, will vary and the dose that is required will vary accordingly. Furthermore, the plasma concentration required in order for one subject to achieve a particular state may depend on other characteristics of the individual, for example, body weight, prior administration, tolerance, etc. One skilled in the art would understand that a given dose of a tryptamine may be converted to one or more other measures of dose.

In some embodiments, it is desirable to provide a dose of the psychedelic tryptamine below a maximum threshold level which induces noticeable perceptual effects. Such treatments may be referred to as “micro-dosing” (see definition herein) or sub-hallucinogenic dosing. Such sub-hallucinogenic dosing has been shown to have positive effects on cognitive processes as well as demonstrable effects on regions in the brain which are involved in affective processes. Accordingly, in some embodiments of the methods of the invention, the composition that is administered includes a dose of the tryptamine below a dose required to induce noticeable perceptual effects, such as hallucinations. However, in these embodiments, it may be desirable to induce a plasma concentration in a subject about a therapeutic level, such as a level whereby these are serotonergic and/or psychotropic effects.

In other therapeutic applications, it is desirable to induce noticeable perceptual effects, such as hallucinogenic, mystical or spiritual effects). Such effects have been proposed to provide enhanced therapeutic effects in combination with psychotherapy. Therefore, in some embodiments, psychotherapy can be provided while the serum level of the psychedelic tryptamine is above a threshold level-being a level whereby there are noticeable perceptual effects, such as hallucinations. Accordingly, in some embodiments of the methods of the invention, the single dosage form pharmaceutical, or the pharmaceutical composition, includes a dose of the psychedelic tryptamine at or above a dose required to induce noticeable perceptual effects, such as hallucinations. Notably, the plasma concentration at which an individual experiences noticeable perceptual effects may vary from individual to individual, and therefore may need to be determined on an individual basis. Alternatively, a threshold plasma concentration, above which there are noticeable perceptual effects, can be based on the observed, calculated or estimated threshold of a population, such as the median or mean threshold observed in a comparative population.

The dosage of the tryptamine will be determined by the desired psychological effect. Traditionally, DMT (for example) has been consumed to induce noticeable perceptual effects, such as hallucinogenic, mystical or spiritual effects. However, tryptamines may provide significant therapeutic utility at dosages below the threshold needed to induce such noticeable perceptual effects. Such doses may be referred to as low doses, micro-doses and sub-hallucinogenic doses, although the noticeable perceptual effects are not limited to hallucinations and may also encompass mystical or spiritual effects. Accordingly, in certain embodiments, the dose of the tryptamine administered is below a threshold required to induce noticeable perceptual effects in a subject. In other embodiments, the dose of the tryptamine administered is below a threshold required to induce hallucinations.

In certain embodiments of the methods and compositions as disclosed herein, the tryptamine is N, N-dimethyltryptamine or DMT. In some embodiments, DMT is provided as a pharmaceutically acceptable salt. In some embodiments, DMT is provided as the fumarate salt. In some embodiments, the amount of DMT or a pharmaceutically acceptable salt thereof that is administered is below a threshold required to induce noticeable perceptual effects. In other embodiments, the amount of DMT or a pharmaceutically acceptable salt thereof that is administered is below a threshold required to induce hallucinations.

While such a “hallucinogenic” threshold may vary, in some embodiments, the composition comprises a dose of DMT or a pharmaceutically acceptable salt thereof that provides a peak plasma concentration of DMT in a subject after administration below a maximum threshold of up to 10 μg/L, up to 11 μg/L, up to 12 μg/L, up to 13 μg/L, up to 14 μg/L, up to 15 μg/L, up to 16 μg/L, up to 17 μg/L, up to 18 μg/L, up to 19 μg/L, up to 20 μg/L, up to 25 μg/L, up to 30 μg/L, up to 35 μg/L, or up to 40 μg/L. In certain embodiments, this serum concentration is below the threshold needed to induce noticeable perceptual effects, such as hallucinations.

In other embodiments, the composition comprises a dose of DMT or a pharmaceutically acceptable salt thereof that results in plasma concentrations in a subject above a desired therapeutic threshold. Such a therapeutic threshold may be one that provides demonstrable serotonergic or psychotropic effects. The desired therapeutic threshold can be determined based on the disorder or disease to be treated and can be determined using known assessment techniques and pre-clinical and clinical trials.

In some embodiments, there is provided a composition comprising DMT or a pharmaceutically acceptable salt thereof that provides a plasma concentration of DMT in a subject after administration above a therapeutic threshold level of at least 1 g/L, at least 2 μg/L, at least 3 μg/L, at least 4 μg/L, at least 5 μg/L, at least 6 μg/L, at least 7 μg/L, at least 8 μg/L, at least 9 μg/L, at least 10 μg/L, at least 11 μg/L, at least 12 μg/L, at least 13 μg/L, at least 14 μg/L, at least 16 μg/L, at least 18 μg/L, or at least 20 μg/L.

Where it may be desirable to keep the plasma concentration of DMT in a subject above the therapeutic level for a prolonged period of time, the prolongation of the elimination half-life can be achieved by controlling the inhibition of systemic monoamine oxidases, increasing the initial dose or extending the delayed release formulation of the DMT. Such modifications in the dose of the tryptamine (and/or the MAOI) will increase the time for which the plasma concentration is above the threshold. Further, where the release of the DMT is extended, the dosage of DMT may be increased in the formulation but the peak plasma level may not increase in a commensurate manner as the total dosage is released over a longer period of time. Accordingly, in some embodiments, the composition comprising DMT or a pharmaceutically acceptable salt thereof provides a plasma concentration of DMT in a subject above the therapeutic threshold for at least 30 minutes, at least 60 minutes, at least 90 minutes, at least 120 minutes, at least 150 minutes, at least 180 minutes, at least 210 minutes, at least at least 240 minutes, at least 270 minutes, at least 300 minutes, and least 330 minutes, at least 360 minutes, at least 390 minutes, at least 420 minutes, at least 450 minutes, at least 480 minutes, at least 510 minutes, at least 540 minutes, at least 570 minutes, or at least 600 minutes.

In some embodiments, there is provided a composition comprising up to 5 mg, up to 6 mg, up to 7 mg, up to 8 mg, up to 9 mg, up to 10 mg, up to 11 mg, up to 12 mg, up to 13 mg, up to 14 mg, up to 15 mg, up to 20 mg, up to 25 mg, or up to 30 mg of DMT, or a pharmaceutically acceptable salt thereof. In some embodiments, the composition comprises a dose DMT, or a pharmaceutically acceptable salt thereof, of up to 0.6 mg/kg, up to 0.5 mg/kg, up to 0.4 mg/kg, up to 0.3 mg/kg, up to 0.2 mg/kg, up to 0.15 mg/kg, up to 0.1 mg/kg, or up to 0.05 mg/kg, when administered to a subject. In some embodiments, the dosage of DMT or a pharmaceutically acceptable salt thereof is selected such that when administered to a subject, the dose does not induce noticeable perceptual effects, such as hallucinations.

The desired dose of DMT may be determined on a subject-to-subject basis, based on an evaluation of an individual's pharmacokinetics for DMT (alone or in combination with the MAOI) and the corresponding plasma concentration. Alternatively, the dosage may be based on the observed, calculated or estimated pharmacokinetics of a population, such as the median or mean pharmacokinetic profile observed in a comparative population.

The use of psychedelics, such as DMT, at higher dosages has been proposed to assist psychological treatment of certain neurodevelopmental, neurological, movement, psychological or physiological, diseases, disorders, syndromes or symptoms by inducing altered perceptual effects. Therefore, in some embodiments, the composition comprises a dosage of DMT or a pharmaceutically acceptable salt thereof above a threshold needed to induce noticeable perceptual effects in a subject. In some embodiments, the dosage of DMT or a pharmaceutically acceptable salt thereof is above the threshold needed to induce hallucinations.

In some embodiments, the composition comprising an amount of DMT or a pharmaceutically acceptable salt thereof provides a plasma concentration of DMT in a subject after administration above a threshold level of at least 12 μg/L, at least 15 μg/L, at least 20 μg/L, at least 25 μg/L, at least 30 μg/L, at least 35 μg/L, at least 40 μg/L, at least 45 μg/L, at least 50 μg/L, at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, or at least 100 μg/L. In some embodiments, the composition comprising DMT or a pharmaceutically acceptable salt thereof provides a plasma concentration of DMT in a subject after administration above a threshold level of at least 50 μg/L, at least 60 μg/L, or at least 70 μg/L, between 50 μg/L to 70 μg/L, between 60 μg/L to 80 μg/L between 50 μg/L to 80 μg/L, between 50 μg/L to 90 μg/L, between 30 μg/L to 60 μg/L, between 30 μg/L to 50 μg/L, between 20 μg/L to 50 μg/L, between 20 μg/L to 40 μg/L, between 10 μg/L to 30 μg/L, between 10 μg/L to 40 μg/L, between 10 μg/L to 50 μg/L, between 10 μg/L to 60 μg/L, between 10 μg/L to 70 μg/L, between 50 μg/L to 80 μg/L, between 10 μg/L to 90 μg/L or between 10 μg/L to 100 μg/L.

In the clinical setting, when a state of altered perception is induced, it may be necessary to monitor or supervise the subject. In addition, it may be advantageous to provide psychotherapy or occupational therapy during this state of altered perception. As such, a time-limited experience is desirable as this allows scheduling of treatment within a clinical setting or a standard clinical rotation. Accordingly, in some embodiments there is provided a composition comprising DMT or a pharmaceutically acceptable salt thereof that provides a plasma concentration of DMT above the threshold level for noticeable perceptual effects in a subject for up to 30 minutes, up to 35 minutes, up to 45 minutes, up to 50 minutes, up to 55 minutes, up to 60 minutes, up to 90 minutes, up to 120 minutes, or up to 150 minutes.

In some embodiments, the composition comprises DMT or a pharmaceutically acceptable salt thereof in an amount of at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 140 mg, at least 160 mg, at least 180 mg, or at least 200 mg. In some embodiments, the composition comprises DMT or a pharmaceutically acceptable salt thereof in an amount to provide a dose of up to 2 mg/kg, up to 1.5 mg/kg, up to 1 mg/kg, up to 0.8 mg/kg, up to 0.6 mg/kg, up to 0.5 mg/kg, up to 0.4 mg/kg, up to 0.3 mg/kg, up to 0.2 mg/kg, up to 0.1 mg/kg, up to 0.05 mg/kg or up to 0.01 mg/kg when administered to a subject. In some forms of these embodiments, the dosage is selected such that when administered to a subject, the dose induces noticeable perceptual effects, such as hallucinations.

In certain embodiments of the methods and compositions as disclosed herein, the tryptamine is 5-methoxy-N,N-dimethyltryptamine (5-MeO-DMT). In some embodiments, 5-MeO-DMT is provided as a pharmaceutically acceptable salt. In some embodiments, the amount of 5-MeO-DMT or a pharmaceutically acceptable salt thereof that is administered is below a threshold required to induce noticeable perceptual effects. In other embodiments, the amount of 5-MeO-DMT or a pharmaceutically acceptable salt thereof that is administered is below a threshold required to induce hallucinations.

While such a “hallucinogenic” threshold may vary, in some embodiments, the composition comprises a dose of 5-MeO-DMT or a pharmaceutically acceptable salt thereof that provides a peak plasma concentration of 5-MeO-DMT in a subject after administration below a maximum threshold of up to 2 μg/L, up, up to 2.5 μg/L, up to 3 μg/L, up to 3.5 μg/L, up to 4 μg/L, up to 4.5 μg/L, up to 5 μg/L, up to 6 μg/L, or up to 7.5 μg/L. In certain embodiments, this plasma concentration is below the threshold needed to induce noticeable perceptual effects, such as hallucinations.

In other embodiments, the composition comprises a dose of 5-MeO-DMT or a pharmaceutically acceptable salt thereof that results in plasma concentrations in a subject above a desired therapeutic threshold. Such a therapeutic threshold may be one that provides demonstrable serotonergic or psychotropic effects. The desired therapeutic threshold can be determined based on the disorder or disease to be treated and can be determined using known assessment techniques and pre-clinical and clinical trials.

In some embodiments, there is provided a composition comprising a dose of 5-MeO-DMT or a pharmaceutically acceptable salt thereof that provides a plasma concentration of 5-MeO-DMT in a subject after administration above a therapeutic threshold level of at least 0.2 g/L, at least 0.3 μg/L, at least 0.4 μg/L, at least 0.5 g/L, at least 0.6 μg/L, at least 0.7 μg/L, at least 0.8 μg/L, at least 0.9 μg/L, at least 1 μg/L, at least 1.5 μg/L, at least 2 μg/L, at least 2.5 μg/L, at least 3 μg/L, or at least 3.5 μg/L.

Where it may be desirable to keep the plasma concentration of 5-MeO-DMT in a subject above the therapeutic level for an extended period of time, the prolongation of the elimination half-life can be achieved by controlling the inhibition of systemic monoamine oxidases, increasing the initial dose or prolongation of the delayed release formulation of the 5-MeO-DMT. Such changes in the dose of the tryptamine and/or the MAOI will increase the time for which the plasma concentration is above the threshold.

Further, where the release of the 5-MeO-DMT is extended, the dosage of 5-MeO-DMT may be increased in the formulation but the peak plasma level may not increase in a commensurate manner as the total dosage is released over a longer period of time. Accordingly, in some embodiments, the composition comprising 5-MeO-DMT or a pharmaceutically acceptable salt thereof provides a plasma concentration of 5-MeO-DMT in a subject above the therapeutic threshold for at least 30 minutes, at least 60 minutes, at least 90 minutes, at least 120 minutes, at least 150 minutes, at least 180 minutes, at least 210 minutes, at least at least 240 minutes, at least 270 minutes, at least 300 minutes, and least 330 minutes, at least 360 minutes, at least 390 minutes, at least 420 minutes, at least 450 minutes, at least 480 minutes, at least 510 minutes, at least 540 minutes, at least 570 minutes, or at least 600 minutes.

The dosage required to achieve the desired plasma concentrations will vary depending on individual pharmacokinetics and the formulation of the single oral dosage form or pharmaceutical composition. Accordingly, in some embodiments, there is provided a composition comprising 5-MeO-DMT or a pharmaceutically acceptable salt thereof in an amount of up to 1 mg, up to 1.5 mg, up to 2 mg, up to 2.5 mg, up to 3 mg, up to 3.5 mg, up to 4 mg, up to 4.5 mg, up to 5 mg, up to 5.5 mg, up to 6 mg, up to 6.5 mg, up to 7 mg, up to 7.5 mg, or up to 10 mg. In some embodiments, the composition comprises 5-MeO-DMT or a pharmaceutically acceptable salt thereof in an amount to provide a dose of up to 0.02 mg/kg, or up to 0.03 mg/kg, or up to 0.04 mg/kg, up to 0.05 mg/kg, up to 0.06 mg/kg, up to 0.07 mg/kg, up to 0.08 mg/kg, up to 0.09 mg/kg, up to 0.1, up to 0.11, or up to 0.125 mg/kg, when administered to a subject. In some forms of these embodiments, the dosage is selected such that when administered to a subject, the dose does not induce noticeable perceptual effects, such as hallucinations.

The desired dose of 5-MeO-DMT may be determined on a subject-to-subject basis, based on an evaluation of an individual's pharmacokinetics for 5-MeO-DMT (alone or in combination with the MAOI) and the corresponding plasma concentration. Alternatively, the dosage may be based on the observed, calculated or estimated pharmacokinetics of a population, such as the median or mean pharmacokinetic profile observed in a comparative population.

The use of high levels of psychedelics, such as 5-MeO-DMT, has been proposed to assist psychological treatment of certain neurodevelopmental, neurological, movement, psychological, diseases, disorders, syndromes or symptoms by inducing altered perceptual effects. Therefore, in some embodiments, the composition comprises a dosage of 5-MeO-DMT or a pharmaceutically acceptable salt thereof above a threshold needed to induce noticeable perceptual effects in a subject. In some embodiments, the dosage of 5-MeO-DMT is above the threshold needed to induce hallucinations.

In some embodiments, the invention provides a composition comprising 5-MeO-DMT or a pharmaceutically acceptable salt thereof in an amount to provide a plasma concentration of 5-MeO-DMT in a subject after administration above a threshold level of at least 2.5 μg/L, 3 μg/L, at least 3.5 μg/L, at least 4 μg/L, at least 4.5 μg/L or at least 5 μg/L, at least 6 μg/L, at least 7 μg/L, at least 8 μg/L, at least 9 μg/L, at least 10 μg/L, at least 11 μg/L, at least 12 μg/L, at least 13 μg/L, at least 14 μg/L, at least 15 μg/L, at least 16 μg/L, at least 17 μg/L, at least 18 μg/L, at least 19 μg/L, at least 20 μg/L, at least 21 μg/L, at least 22 μg/L, at least 23 μg/L, at least 24 μg/L, or at least 25 μg/L. In a preferred embodiment, there is provided a composition comprising 5-MeO-DMT or a pharmaceutically acceptable salt thereof in an amount that provides a plasma concentration of 5-MeO-DMT in a subject after administration above a threshold level of at least 12.5 μg/L, at least 15 μg/L, at least 17.5 μg/L, or between 12.5 μg/L to 17.5 μg/L. In another preferred embodiment, there is provided a composition comprising 5-MeO-DMT or a pharmaceutically acceptable salt thereof in an amount that provides a plasma concentration of 5-MeO-DMT in a subject after administration above a threshold level of at least 10 μg/L, at least 12 μg/L, or at least 14 μg/L, or between 10 μg/L to 14 μg/L.

In the clinical setting, when a state of altered perception is induced, it may be necessary to monitor or supervise the subject. In addition, it may be advantageous to provide psychotherapy or occupational therapy during this state of altered perception. As such, a time-limited experience is desirable as this allows scheduling of treatment within a clinical setting or a standard clinical rotation. Accordingly, in some embodiments the composition comprising 5-MeO-DMT or a pharmaceutically acceptable salt thereof provides a plasma concentration of 5-MeO-DMT above the threshold level for noticeable perceptual effects in a subject for up to 30 minutes, up to 35 minutes, up to 45 minutes, up to 50 minutes, up to 55 minutes, up to 60 minutes, or up to 90 minutes.

In some embodiments, the present invention provides a composition comprising 5-MeO-DMT or a pharmaceutically acceptable salt thereof in an amount of at least 1.5 mg, at least 2 mg, at least 3 mg, at least 4 mg, at least 5 mg, at least 6 mg, at least 7 mg, at least 8 mg, at least 9 mg, at least 10 mg, at least 11 mg, at least 12 mg, or at least 13 mg, at least 14 mg, at least 15 mg, at least 16 mg, at least 17 mg, at least 18 mg, at least 19 mg, at least 20 mg, at least 22 mg, at least 24 mg, at least 26 mg, at least 28 mg, at least 30 mg, at least 35 mg, at least 40 mg, or at least 45 mg. In some embodiments, the composition comprises 5-MeO-DMT or a pharmaceutically acceptable salt thereof in an amount to provide a dose of up to 0.65 mg/kg, 0.6 mg/kg, 0.55 mg/kg, 0.5 mg/kg, up to 0.45 mg/kg, 0.4 mg/kg, 0.35 mg/kg, up to 0.3 mg/kg, 0.25 mg/kg, up to 0.2 mg/kg, 0.15 mg/kg, or up to 0.1 mg/kg, when administered to a subject. In some forms of these embodiments, the dosage is selected such that when administered to a subject, the dose induces noticeable perceptual effects, such as hallucinations.

The desired dose of psilocin, or a prodrug equivalent thereof, may be based on evaluation of an individual's pharmacokinetics for psilocin (alone or in combination with the MAOI) and the corresponding plasma concentration. Alternatively, the dosage may be based on the observed, calculated or estimated pharmacokinetics of a population, such as the median or mean pharmacokinetic profile observed in a comparative population.

Psilocin (and not psilocybin) is primarily responsible for the psychedelic effect of magic mushrooms, not psilocybin.

Accordingly, the desired therapeutic ranges and thresholds herein (as they relate to psilocin and psilocybin) will be defined as the plasma concentration of psilocin. One skilled in the art will be capable of calculating the dosage of a prodrug of psilocin (such as psylocibin) to determine an equivalent plasma concentration taking into account known bioavailability and pharmacokinetics compared to the equivalent dosage of pure psilocin.

As individual dosages may vary due to body weight and MAO expression, in some embodiments of the invention, there is provided a composition comprising psilocin or a pharmaceutically acceptable salt thereof in an amount to provide a peak plasma concentration of psilocin in a subject after administration below a maximum threshold of up to 6 μg/L, up to 5 μg/L, up to 4 μg/L, up to 2 μg/L, up to 1 μg/L, or up to 0.5 μg/L. In some embodiments, the plasma concentration is below the threshold needed to induce noticeable perceptual effects, such as hallucinations.

Further, it may be desirable to provide a composition which results in plasma concentrations above a desired therapeutic threshold. Such a therapeutic threshold may be one that provides demonstrable serotonergic or psychotropic effects. The desired therapeutic threshold can be determined based on the disorder or disease to be treated and can be determined using known assessment techniques and pre-clinical and clinical trials.

In some embodiments, there is provided a composition comprising psilocin or a pharmaceutically acceptable salt thereof in an amount to provide a plasma concentration of psilocin in a subject after administration above a therapeutic threshold of at least 0.2 μg/L, at least 0.3 μg/L, at least 0.4 μg/L, at least 0.5 μg/L, at least 0.6 μg/L, at least 0.7 μg/L, at least 0.8 μg/L, at least 0.9 μg/L, at least 1.0 μg/L, at least 1.2 μg/L, at least 1.4 μg/L, at least 1.6 μg/L, at least 1.8 μg/L, or at least 2.0 μg/L.

Where it may be desirable to keep the plasma concentration of psilocin in a subject above the therapeutic level for an extended period of time, the prolongation of the elimination half-life can be achieved by controlling the inhibition of systemic monoamine oxidases, increasing the initial dose or prolongation of the delayed release formulation of the psilocin (or the prodrug of psilocin). Such changes in the dose of the tryptamine and/or MAOI will increase the time for which the concentration of psilocin is above the threshold. Further, where the release of the psilocin is extended, the dosage of psilocin may be increased in the composition, but the peak plasma level may not increase in a commensurate manner as the total dosage is released over a longer period of time. Accordingly, in some embodiments, the composition comprises psilocin or a pharmaceutically acceptable salt thereof in an amount that provides a plasma concentration of psilocin above the therapeutic threshold for at least for at least 30 minutes, at least 60 minutes, at least 120 minutes, at least 150 minutes, at least 180 minutes, at least 210 minutes, at least 240 minutes, at least 270 minutes, at least 300 minutes, at least 330 minutes, at least 360 minutes, at least 390 minutes, at least 420 minutes, at least 450 minutes, at least 480 minutes, at least 510 minutes, at least 540 minutes, at least 570 minutes, or at least 600 minutes.

In some embodiments of the invention, there is provided a composition comprising psilocin or a pharmaceutically acceptable salt thereof in an amount of up to 4 mg, up to 3 mg, up to 2 mg, or up to 1 mg, or up to 0.5 mg, or a prodrug equivalent thereof. In some embodiments, the composition comprises psilocin or a pharmaceutically acceptable salt thereof, or a prodrug equivalent thereof, in an amount to provide a dose of up to 5 μg/kg, up to 7.5 μg/kg, up to 10 μg/kg, up to 12.5 μg/kg, up to 15 μg/kg, up to 17.5 μg/kg, up to 20 μg/kg, up to 22.5 μg/kg or up to 25 μg/kg. In some forms of these embodiments, the dosage is selected such that when administered to a subject, the dose does not induce noticeable perceptual effects, such as hallucinations.

The desired dose of psilocin, or a prodrug equivalent thereof, may be based on evaluation of an individual's pharmacokinetics for psilocin (alone or in combination with the MAOI) and the corresponding plasma concentration. Alternatively, the dosage may be based on the observed, calculated or estimated pharmacokinetics of a population, such as the median or mean pharmacokinetic profile observed in a comparative population.

As described above, the compositions disclosed herein comprise a monoamine oxidase inhibitor. In certain embodiments, the MAOI is moclobemide. The dose of moclobemide that is provided in a composition for administration may be varied in accordance with the requirements of a particular subject. As disclosed herein, where the composition comprises both a MAOI and a tryptamine, the MAOI is formulated for immediate release, such that inhibition of the monoamine oxidase (specifically, monoamine oxidase A) in a subject maintains and/or increases the plasma concentration of the tryptamine in the same subject. In certain embodiments, the composition comprises a MAOI in an amount to regulate the peak plasma concentration of the tryptamine in a subject. In other embodiments, the composition comprises a MAOI in an amount to increase the duration in which the plasma concentration of a tryptamine is maintained above a given threshold.

In some embodiments, the composition comprises up to 50 mg, up to 100 mg, up to 150 mg, up to 200 mg, up to 250 mg, up to 300 mg, up to 350 mg, up to 400 mg, up to 450 mg, up to 500 mg, up to 550 mg, or up to 600 mg of Moclobemide.

In some embodiments, the composition comprises 75 mg or less, 100 mg or less, 150 mg or less, 200 mg or less, 250 mg or less, 300 mg or less, 350 mg or less, 400 mg or less, 450 mg or less, 500 mg or less, 550 mg or less, 600 mg or less, or 650 mg or less of Moclobemide.

In some embodiments, the invention provides an immediate release sublingual or buccal dose of Moclobemide. Such a dose of Moclobemide can be used alone, or it may form part of a single dosage form of Moclobemide and a psychedelic tryptamine. In some embodiments, the sublingual or buccal dosage form is a solid, film gel film or troche dosage form. In some embodiments, the dosage form is in the form of a tablet. In some embodiments, the dosage form is in the form of a film. In some embodiments, the dosage form is in the form of a gel. In some embodiments, the sublingual or buccal dosage form comprises 75 mg or less, 100 mg or less, 150 mg or less, 200 mg or less, 250 mg or less, 300 mg or less, 350 mg or less, 400 mg or less, 450 mg or less, 500 mg or less, 550 mg or less, 600 mg or less, or 650 mg or less of Moclobemide. In some embodiments, the sublingual or buccal dosage form comprises up to 50 mg, up to 100 mg, up to 150 mg, up to 200 mg, up to 250 mg, up to 300 mg, up to 350 mg, up to 400 mg, up to 450 mg, up to 500 mg, up to 550 mg, or up to 600 mg of Moclobemide.

Further provided is the use of a psychedelic tryptamine and a MAOI in the preparation of a medicament for the treatment of a neurodevelopmental, neurological, movement, psychological or physiological, disease, disorder, syndrome or symptom in a subject. Also provided is the use of a composition (such as those described herein) in the preparation of a medicament for the treatment of a neurodevelopmental, neurological, movement, psychological or physiological, disease, disorder, syndrome or symptom in a subject.

### EXAMPLES

The invention is further described and illustrated in the following examples. The examples are only for the purpose of describing particular embodiments of the invention and are not intended to be limiting with respect to the above description and the scope of the invention as claimed in this application or future applications claiming priority from this application.

### General Experimental Details

High-performance liquid chromatography (HPLC) was performed on a Shimadzu HPLC system with a CBM-20A communication BUS module, LC-20AD pump system, SIL-20A HT auto sample injector, SPD 20A UV-vis detector and CTO-20A column oven.

### Example 1—Preparation of a Delayed Release Formulation of DMT Fumarate

20.16 g of Surelease E-7-19040 and 0.89 g of Opadry OY-29020 were added to 29.29 g of deionized water and mixed to provide a coating solution. The amount of the coating applied to be applied to a sample of DMT fumarate was calculated as a percentage of the weight of DMT fumarate that is to be coated (% WG). Coating of DMT fumarate was performed at 35° C. with an atomiser at a pressure of between 10 to 12.5 psi. The coated sample of DMT fumarate was allowed to dry.

### Example 2—Determination of the Release Profile of a Delayed Release Formulation of DMT Fumarate

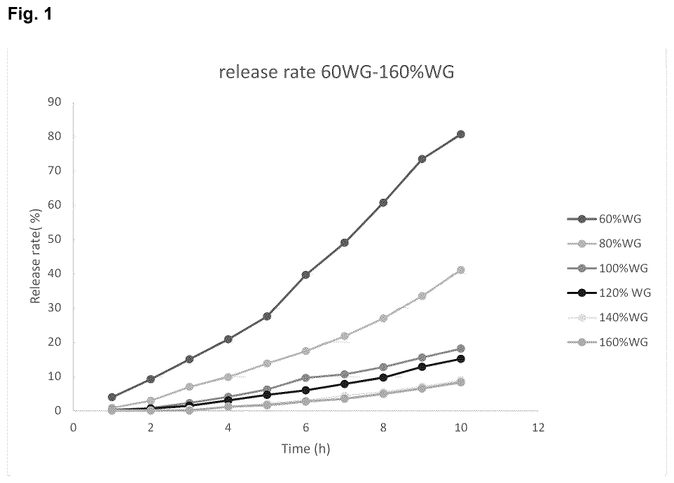

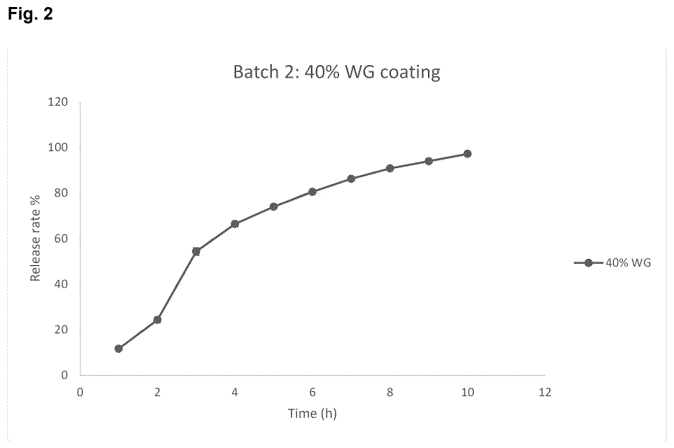

A Basket Apparatus (Apparatus 1, Sotax) according to the United States Pharmacopeia (USP) was employed to assess the release behaviour of the delayed release formulation of DMT fumarate. A 150 mg sample was accurately weighed and filled into the basket. Each basket was put into a vessel containing 300 mL of 0.1 M HCl. The speed of the paddle was 75 rpm and the temperature of the solution was 37±0.5° C. After 2 h, 1 mL samples were withdrawn, and then 600 mL of 0.1 M Na2HPO4 pre-equilibrated to 37±0.5° C. was added to each vessel. The pH of the solution was adjusted to pH 6.8±0.05. 1 mL samples were withdrawn at every hour. The medium was kept at a constant volume by refilling it with fresh buffer solution. The withdrawn samples subsequently were filtered through a 0.45 μm filter and assayed for the dissolved DMTF concentration by HPLC as described above. The percentages of the DMT fumarate released with time are found in Table 1. Charts showing the release profile can be found in FIGS. 1 and 2.

TABLE 1

| | | | | | | |
|---|---|---|---|---|---|---|
| Coating % | 60 | 80 | 100 | 120 | 140 | 160 |
| Sample | 3512.65 | 3350.62 | 3815.999 | 3175.22 | 3353.96 | 2849.48 |
| weight (μg) | | | | | | |

% DMT fumarate released

| | | | | | | | |
|---|---|---|---|---|---|---|---|
| Time | 1 | 3.9 | 0.84 | 0.21 | 0 | 0 | 0 |
| (h) | 2 | 9.13 | 2.94 | 0.78 | 0.55 | 0 | 0 |
| | 3 | 14.96 | 6.96 | 2.23 | 1.39 | 0.39 | 0 |
| | 4 | 20.78 | 9.81 | 4.02 | 2.95 | 1.17 | 1.15 |
| | 5 | 27.43 | 13.77 | 6.16 | 4.58 | 2.19 | 1.55 |
| | 6 | 39.54 | 17.37 | 9.55 | 5.92 | 3.16 | 2.62 |
| | 7 | 48.9 | 21.72 | 10.58 | 7.76 | 4.49 | 3.45 |
| | 8 | 60.58 | 26.92 | 12.69 | 9.61 | 5.53 | 4.86 |
| | 9 | 73.3 | 33.44 | 15.48 | 12.76 | 7.15 | 6.42 |
| | 10 | 80.53 | 41.04 | 18.07 | 15.07 | 8.79 | 8.21 |

### Example 3—HPLC Analysis of a Delayed Release Formulation of DMT Fumarate

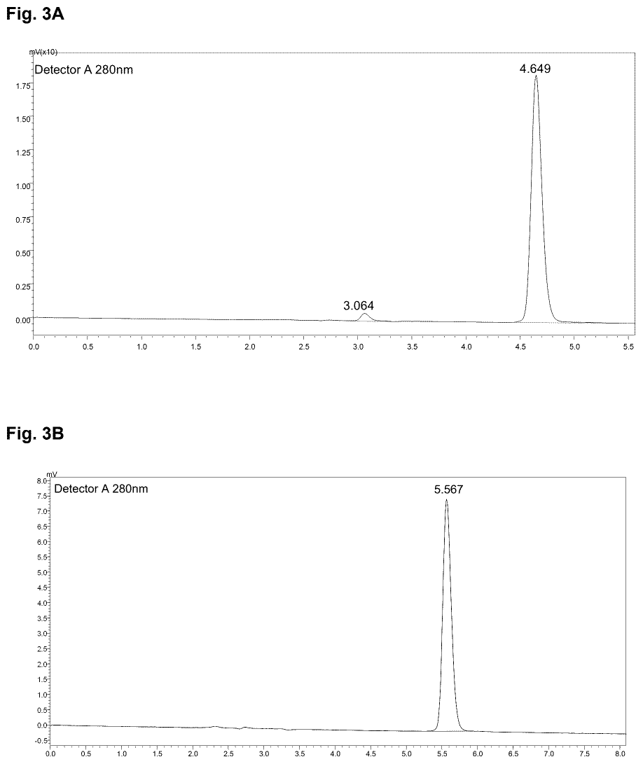

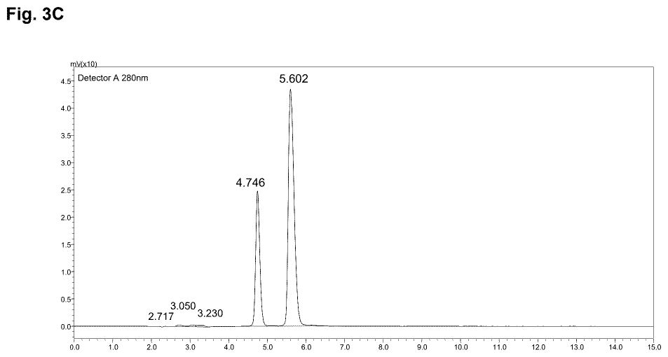

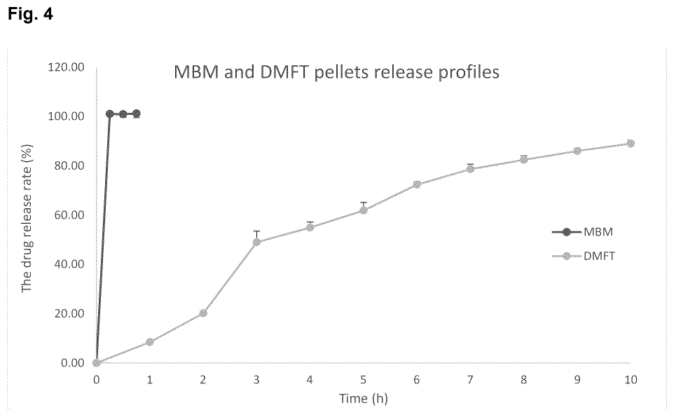

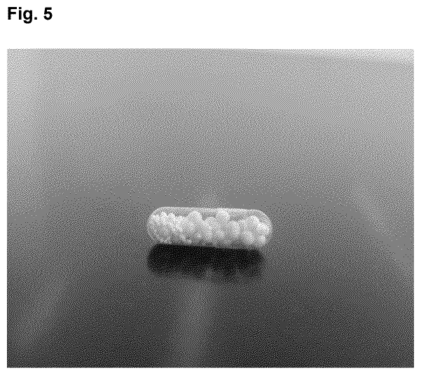

The delayed release formulation of DMT fumarate prepared according to the process as described above was allowed to disintegrate. HPLC analysis of the resulting composition used a Kinetex 5 μm, Phenyl-Hexyl 100 Å column at 40° C., mobile phase of phosphate buffer solution:acetonitrile (90:10 v/v), and UV detection at 277 nm. Retention time of DMT fumarate was 17±1 min (see FIG. 3A).

### Example 4—Development of an Immediate Release Formulation of Moclobemide

Tablet formulations without moclobemide were prepared for evaluation of hardness/mechanical strength, weight and friability (see Table 2).

TABLE 2

| | | | | Weighed |
|---|---|---|---|---|
| | | Weight | Weight | amount |
| Formula- | | (1 tablet) | (5 tablets) | (5 tablets) |
| tion | Ingredients | (mg)* | (mg)* | (mg)*** |
| F1 | Mannitol | 100 | 500 | 502.6 |
| | Magnesium Stearate | 17.5 | 87.5 | 87.5 |
| | Hydroxyethyl- | 40 | 200 | 201.8 |
| | cellulose | | | |
| | Starch | 40 | 200 | 200 |
| Total weight (mg) | | 197.5 | | |
| F2 | Mannitol | 100 | 500 | 500.9 |
| | Magnesium Stearate | 17.5 | 87.5 | 88.3 |
| | Hydroxyethyl- | 45 | 225 | 225.4 |
| | cellulose | | | |
| | Starch | 45 | 225 | 225.9 |
| Total weight (mg) | | 207.5 | | |
| F3 | Mannitol | 100 | 500 | 507.5 |
| | Magnesium Stearate | 17.5 | 87.5 | 88 |
| | Microcrystalline | 50 | 250 | 251.5 |
| | cellulose | | | |
| | Kiccolate ND-2HS | 50 | 250 | 251.9 |
| Total weight (mg) | | 217.5 | | |
| F4 | Mannitol | 100 | 500 | 505.7 |
| | Magnesium Stearate | 17.5 | 87.5 | 88.5 |
| | Microcrystalline | 70 | 350 | 350 |
| | cellulose | | | |
| | Kiccolate ND-2HS | 30 | 150 | 150 |
| Total weight (mg) | | 217.5 | | |
| F5 | Mannitol | 150 | 750 | 750 |
| | Magnesium Stearate | 17.5 | 87.5 | 88.7 |
| | Kiccolate ND-2HS | 70 | 350 | 351.2 |
| | Microcrystalline | 30 | 150 | 151.4 |
| | cellulose | | | |

| | |
|---|---|
| Total weight (mg) | 267.5 |

*Theoretical weight;
***Actual weight

Tablet formulations F1 to F5 were evaluated for weight and hardness/mechanical strength. Formulations that passed hardness testing proceeded to friability testing (see Table 3).

TABLE 3

| ID | Weight (mg) | Hardness (kg) | Verdict |
|---|---|---|---|
| F1 | 181 | 0.28 | Unsuccessful |
| F2 | 215 | 1.22 | Unsuccessful |
| F3 | 218 | 4.58 | Successful |
| F4 | 190 | 4.37 | Successful |
| F5 | 204 | 3.42 | Successful |

Friability (100 cycles) was tested using the EP-2 Friabilator (USP). Formulations that passed friability testing (i.e. friability ≤1%) proceeded to disintegration testing (see Table 4).

TABLE 4

| | Initial | Final weight | | |
|---|---|---|---|---|
| | weight | (after test) | | |
| ID | (mg) | (mg) | Remarks | Verdict |
| F3 | 417 | 414 | Excellent friability (0.72%) | Successful |
| F4 | 388 | 382 | Failed friability testing | Unsuccessful |
| | | | (1.55%) | |
| F5 | 402 | 405 | Excellent friability (0.75%) | Successful |

Disintegration testing of tablet formulations F3 and F5 was performed, however both formulations failed disintegration tests.

### Example 5—Further Development of a Sublingual Formulation of Moclobemide

Since none of the formulations in Example 4 provided the required characteristics for an immediate release formulation, additional formulations were prepared in accordance with Table 5.

TABLE 5

| | | | | Weighed |
|---|---|---|---|---|
| | | Amount | Amount | amount |
| | | (1 tablet) | (10 tablets) | (10 tablets) |
| ID | Ingredient | (mg)* | (mg)* | (mg)*** |
| F6 | Mannitol | 100 | 1000 | 1012 |
| | Magnesium Stearate | 17.5 | 175 | 175.1 |
| | Kiccolate ND-2HS | 10 | 100 | 100 |
| | Microcrystalline | 43 | 430 | 429.9 |
| | cellulose | | | |
| | Kollidon SR | 54 | 540 | 539.9 |
| Total weight (mg) | | 224.5 | 2245 | 2256.9 |
| F7 | Mannitol | 100 | 1000 | 1044 |
| | Magnesium Stearate | 17.5 | 175 | 175.5 |
| | Kiccolate ND-2HS | 10 | 100 | 100.2 |
| | Microcrystalline | 43 | 430 | 430.1 |
| | cellulose | | | |
| | Kollidon SR | 54 | 540 | 540.8 |
| | 2-Hydroxypropyl-β- | 10 | 100 | 100.5 |
| | cyclodextrin | | | |
| Total weight (mg) | | 234.5 | 2345 | 2391.1 |
| F8 | Mannitol | 100 | 1000 | 1000.1 |
| | Magnesium Stearate | 17.5 | 175 | 175.3 |
| | Kiccolate ND-2HS | 10 | 100 | 100.7 |
| | M | 43 | 430 | 430.2 |
| | Kollidon SR | 54 | 540 | 540.8 |
| Total weight (mg) | | 224.5 | 2245 | 2247.1 |
| F9 | Mannitol | 100 | 1000 | 1005.4 |
| | Magnesium Stearate | 17.5 | 175 | 176.5 |
| | Kiccolate ND-2HS | 10 | 100 | 102.5 |
| | Microcrystalline | 40 | 400 | 405.1 |
| | cellulose | | | |
| | PVP30 | 60 | 600 | 601 |
| Total weight (mg) | | 227.5 | 2275 | 2290.5 |
| F10 | Mannitol | 100 | 1000 | 1001.1 |
| | Magnesium Stearate | 17.5 | 175 | 173.8 |
| | Kiccolate ND-2HS | 10 | 100 | 100 |
| | Microcrystalline | 20 | 200 | 202.5 |
| | cellulose | | | |
| | PVP30 | 30 | 300 | 300 |
| Total weight (mg) | | 177.5 | 1775 | 1776.4 |
| F11-1 | Mannitol | 85 | 1700 | 1701.6 |
| | Magnesium Stearate | 0.25 | 5 | 5.2 |
| | Microcrystalline | 15 | 300 | 303.8 |
| | cellulose | | | |
| | Talc | 0.75 | 15 | 15.5 |

| | | | |
|---|---|---|---|
| Total weight (mg) | 101 | 2020 | 2026.1 |

*Theoretical weight;
***Actual weight

The formulation denoted F11-1 in Table 5 passed all physicochemical tests, however the excipients are not water soluble. In order to provide formulations that contain water soluble excipients, magnesium stearate and microcrystalline cellulose were substituted with Gelucire 48/16 and anhydrous lactose, respectively, for further development. These additional formulations are denoted F12 to F15 (see Table 6).

TABLE 6

| | | Amount | Amount | Weighed amount |
|---|---|---|---|---|
| | | (1 tablet) | (10 tablets) | (10 tablets) |
| ID | Ingredient | (mg)* | (mg)* | (mg)*** |
| F12 | Mannitol | 85 | 1700 | 1701.6 |
| | Gelucire 48/16 | 1 | 20 | 5.2 |
| | Anhydrous lactose | 15 | 300 | 303.8 |
| Total weight (mg) | | 101 | 2020 | 2026.1 |
| F13 | Mannitol | 80 | 1600 | 1600 |
| | Gelucire 48/16 | 2 | 40 | 40.7 |
| | Anhydrous lactose | 20 | 400 | 406.8 |
| Total weight (mg) | | 102 | 2040 | 2047.5 |
| F14 | Mannitol | 75 | 1500 | 1502.2 |
| | Gelucire 48/16 | 5 | 100 | 100.6 |
| | Anhydrous lactose | 25 | 500 | 501.8 |
| Total weight (mg) | | 102 | 2100 | 2104.6 |
| F15 | Mannitol | 80 | 1600 | 1600 |
| | Gelucire 48/16 | 5 | 100 | 102.7 |
| | Anhydrous lactose | 25 | 500 | 502 |

| | | | |
|---|---|---|---|
| Total weight (mg) | 110 | 2100 | 2205.3 |

*Theoretical weight;
***Actual weight

The formulation denoted as F15 in Table 6 passed all physicochemical testing.

Tablets containing 50 mg of moclobemide were prepared. For the formulations denoted as FM1 to FM3, moclobemide and mannitol were ground together for 30 to 60 minutes before mixing with the remaining excipients. All ingredients were then sieved through a 350 μm sieve three times before a tablet was prepared (Table 7).

TABLE 7

| | | Amount | Amount | Weighed amount |
|---|---|---|---|---|
| | | (1 tablet) | (2 tablets) | (2 tablets) |
| ID | Composition | (mg)* | (mg)* | (mg)*** |
| FM1 | Moclobemide | 50 | 100 | 102.4 |
| | Mannitol | 85 | 170 | 171.8 |
| | Microcrystalline | 0.25 | 0.5 | 0.903 |
| | cellulose | | | |
| | Talc | 0.75 | 1.5 | 3.033 |
| Total weight (mg) | | 136 | 272 | 278.136 |
| FM2 | Moclobemide | 50 | 200 | 200.71 |
| | Mannitol | 50 | 200 | 200.88 |
| | MCC | 0.25 | 1 | 1.125 |
| Total weight (mg) | | 100.25 | 401 | 402.715 |
| FM3 | Moclobemide | 50 | 200 | 200.07 |
| | Mannitol | 50 | 200 | 200.23 |
| | Kiccolate ND -2HS | 10 | 40 | 40.6 |
| Total weight (mg) | | 110 | 440 | 440.9 |
| FM4 | Moclobemide | 50 | 100 | 100.15 |
| | Mannitol | 50 | 100 | 100.15 |
| | Kiccolate ND -2HS | 5 | 10 | 10.398 |
| Total weight (mg) | | 105 | 210 | 210.698 |
| FM5 | Moclobemide | 250 | 250.7 | 50.02 |
| | Mannitol | 200 | 200.2 | 39.94 |
| | Kiccolate ND -2HS | 25 | 25.1 | 5.008 |
| | Polyplasdone XL-10 | 25 | 25.2 | 5.03 |

| | | | |
|---|---|---|---|
| Total weight (mg) | 500 | 501.2 | 100 |

Formulations denoted F1 to F5 were evaluated for weight (and weight variation), hardness, friability and disintegration. Only formulation FM5 passed all testing and showed sufficient disintegration (see Table 8).

TABLE 8

| Parameter | Results | Limitation | Pass/Fail |
|---|---|---|---|
| Weight Variation | 94.3-103.6 mg | Variation ± 10% | Passed |
| | (3.3-5.96%) | | |
| Hardness | 2.41-3.21 | 2-3 kgf (min) | Passed |
| Friability | 0.68% | ≤1% | Passed |
| Disintegration (6 tablets) | 12-14 s | ≤120 s | Passed |

Tablets of formulation FM5 containing moclobemide were subjected to dissolution testing in RO water in accordance with USP (see Table 9). All tablets met the required dissolution parameters, i.e. ≥85% dissolution within 15 minutes.

TABLE 9

| | Tablet | Moclobemide | Dissolution % | | |
|---|---|---|---|---|---|
| ID | weight (mg) | (mg) | 5 mins | 10 mins | 15 mins |
| T1 | 98.9 | 49.46 | 97.15 | 114.56 | 119.69 |
| T2 | 100.4 | 50.22 | 93.12 | 119.62 | 123.36 |
| T3 | 109.8 | 55.11 | 84.78 | 104.03 | 123.47 |

Tablets of formulation FM5 containing moclobemide were then subjected to further dissolution testing in a simulated saliva fluid (see Table 10). All tablets met the required dissolution parameters, i.e. ≥85% dissolution within 15 minutes.

TABLE 10

| | Tablet | Moclobemide | Dissolution % | | |
|---|---|---|---|---|---|
| ID | weight (mg) | (mg) | 5 mins | 10 mins | 15 mins |
| FM5D | 106.4 | 53.18 | 96.58 | 99.15 | 101.29 |
| FM5E | 101.8 | 50.88 | 101.86 | 104.57 | 101.82 |
| FM5F | 104.7 | 52.33 | 95.56 | 96.91 | 99.19 |

### Example 6—Preparation of an Immediate Release Formulation of Moclobemide

For each of the formulations described above, moclobemide (where present) was combined with one or more excipients and the mixture was shaken. The mixture was then sieved to provide particles with a given mesh size. Water was then added to provide a mixture with sufficient properties for tablet extrusion. The tablets were then dried.

### Example 7—HPLC Analysis of Moclobemide

HPLC analysis of a composition comprising moclobemide formulated for immediate release used a C18 5 μm, 4.6×250 mm at 30° C., mobile phase of 25% MeCN at pH 2.73, and UV detection at 239 nm. Retention time of moclobemide was 3.35 min (see FIG. 3B).

### Example 8—HPLC Analysis of Single Dosage Form

HPLC analysis of a composition comprising both DMT fumarate formulated for delayed release and moclobemide formulated for immediate release used a C18 5 μm, 4.6×250 mm at 25° C., mobile phase of phosphate buffer solution:MeOH:MeCN (70:10:20, v:v:v), and UV detection at 280 nm. Retention times of DMT fumarate and moclobemide were 4.76±0.01 min and 5.66±0.05 min, respectively (see FIG. 3C).

### Example 9—Animal Pharmacokinetic Study and Behavioural Studies

An in vivo pharmacokinetic and behavioural study may be performed using methodology outlined below.

A total of 30 Sprague Dawley Rats divided into 10 treatment groups (3 per group) will be treated with Moclobemide and one of DMT or psilocybin (as set out in Table 11) spaced 120 minutes apart to simulate the time release of the single oral dosage form.

TABLE 11

| | Moclobemide | DMT | Psilocybin | No. | PK |
|---|---|---|---|---|---|
| Group | (mg/kg) | (mg/kg) | (mg/kg) | Rats | Sample |
| 1 | 0 | 0.05 | 0.007 | 3 | 12 time |
| 2 | 0.35 | 0.05 | 0.007 | 3 | points |
| 3 | 0.53 | 0.05 | 0.007 | 3 | over |
| 4 | 1.07 | 0.05 | 0.007 | 3 | 24 hrs |
| 5 | 2.14 | 0.05 | 0.007 | 3 | |
| 6 | 1.07 | 0 | 0 | 3 | |
| 7 | 1.07 | 0.015 | 0.0007 | 3 | |
| 8 | 1.07 | 0.03 | 0.0017 | 3 | |
| 9 | 1.07 | 0.04 | 0.0035 | 3 | |
| 10 | 1.07 | 0.23 | 0.01 | 3 | |

Note:
Rats will be treated with either Moclobemide and DMT or Moclobemide and Psilocybin.

Blood can be collected via the femoral artery cannula at 12 time points over 24 hours, before plasma is isolated and stored at −80° C. before being analysed. The collected samples can be analysed for plasma concentration of Moclobemide and DMT or Psilocybin with standard pharmacokinetic parameters calculated by non-compartmental methods. This data will indicate the plasma concentration of DMT or Psilocybin over time as well as the time course of Moclobemide.

### Example 10—Behavioural Studies

Upon acquisition, timed pregnant female Sprague Dawley rats were visually inspected and immediately transferred to their home cage (individually ventilated cages (IVCs) supplied by LAS). Animals were kept in a reversed 12:12 hr light/dark schedule (lights on at 6 am) and had ad libitum access to food and water and with environmental enrichment for each cage.

### i) Day 1-7 (Acclimation)

The animals had seven days to acclimatise to their new environment during which time they were monitored daily with minimal handling to minimise stress and anxiety while they are pregnant. Timed pregnant females were due to give birth >1 week after arrival to ensure they have enough time to acclimate to the environment before handling them after the pups are born.

### ii) Day 8 to Day 36 (Behavioural Model Initiation)

Model implemented.

iii) Day 36 to Day 66 (Administration of Drugs)

Following weaning, rats were used to trial the administration routes. All drugs were administered once every 3rd day in order to prevent tolerance against psychedelic compounds. The selected time frame of the pilot treatment (up to 1 month) is to ensure any possible effects may be detected. A 1-month timeframe will allow atotal of 10 injections to be performed, with research suggesting that many compounds that cause irritation do so within 7 injections.

During this period, monitoring of animals occurred twice a day for the first week of treatment, and then reduced from two times daily to once every three days to coincide with the administration of the compounds. Drug treatment was performed over a period of up to 1 month.

### iv) Preparation of the Compounds

Solutions of DMT, 5-MeO-DMT, psilocybin and moclobemide were prepared daily in purified 0.9% NaCl filtered using a 0.2 μm syringe filter. Where required, a standard suspension vehicle [SSV; 0.9% (w/v) NaCl, 0.5% (w/v) Na-carboxymethylcellulose (medium viscosity), 0.5% (v/v) benzyl alcohol, and 0.4% (v/v) Tween-80] may be used, along with other approved and commonly used and safe vehicles, including propylene glycol, propylene glycol 400, <10% DMSO total vehicle volume and <10% Ethanol total vehicle volume. Where drugs are delivered orally, the standard vehicle was diluted in 3 parts condensed milk (v/v) to 10 parts drug solution to form the sweetened milk solution.

### v) Day 68-69 (Culling and Post-Mortem)

Once the trial was completed, rats were euthanised by exposure to isoflurane (5%) in an induction chamber and subsequently prepared for perfusion. A needle was used to puncture the heart and a blood sample was taken. The heart was perfused with 0.1 M phosphate-buffered saline (PBS; 0.1 M sodium dihydrogen phosphate monohydrate, 0.1 M disodium hydrogen phosphate dehydrate, 0.9% w/v sodium chloride; all compounds from Sigma-Aldrich, AUS), pH 7.4. Once perfusion was completed, a secondary euthanasia was performed (decapitation) to ensure the rat is deceased. Whole intact brains were isolated by dissection, with further processing dependent on subsequent experiments.

Table 12 shows the doses of tryptamine (i.e. DMT or psilocybin) and moclobemide (Moc) administered to each group of mice (n=6 for each) during time periods T1, T2, T3 and T4 of the trial protocol. Half of each cohort received 10 days of exercise. Control groups received vehicle only.

TABLE 12

| | Doses | | | |
|---|---|---|---|---|
| Group | T1 | T2 | T3 | T4 |
| 1 | Control | Control | Control | Control |
| 2 | 1.5 mg/kg DMT | 1 mg/kg DMT | 0.5 mg/kg DMT | 2 mg/kg DMT |
| 3 | 2 mg/kg Psil. | 1 mg/kg Psil. | 0.5 mg/kg Psil. | 2 mg/kg Psil. |
| 4 | 1.5 mg/kg DMT, | 1 mg/kg DMT, | 0.5 mg/kg DMT, | 2 mg/kg DMT, |
| | 10 mg/kg Moc | 10 mg/kg Moc | 10 mg/kg Moc | 10 mg/kg Moc |
| 5 | 2 mg/kg DMT, | 1 mg/kg DMT. | 0.5 mg/kg DMT. | 2 mg/kg DMT, |
| | 10 mg/kg Moc | 10 mg/kg Moc | 10 mg/kg Moc | 10 mg/kg Moc |

The head shake response in each rat was determined over a period of 30 minutes after administration of the required dose of a tryptamine (DMT or psilocybin), either with or without Moclobemide (see Tables 13 to 15). The number of head shakes in rats where only either DMT or psilocybin was administered was higher than the control rats and those that were also given Moclobemide. Administration of Moclobemide (i.e. a MAOI) with a tryptamine resulted in less vigorous head shakes in the rats over the observation period. This shows that the administration of a MAOI together with a tryptamine provides a more favourable patient outcome where administration of a tryptamine is required.

TABLE 13
Head shake response at 2 mg/kg Psilocybin and 1.5 mg/kg DMT

| | 0-10 | 10-20 | 20-30 | Head shake |
|---|---|---|---|---|
| | Minutes | Minutes | Minutes | per rat |
| Control | 2 | 1 | 1 | 0.6 |
| MOC + DMT | 9 | 3 | 4 | 2.6 |
| MOC + Psilo | 31 | 16 | 7 | 18 |

TABLE 14
Head shake response at 1 mg/kg Psilocybin and 1 mg/kg DMT

| | 0-10 | 10-20 | 20-30 | Head shake |
|---|---|---|---|---|
| | Minutes | Minutes | Minutes | per rat |
| Control | 2 | 3 | 0 | 0.8 |
| MOC + DMT | 11 | 2 | 5 | 3 |
| MOC + Psilo | 23 | 22 | 4 | 8.16 |

TABLE 15
Head shake response at 0.5 mg/kg Psilocybin and 0.5 mg/kg DMT

| | 0-10 | 10-20 | 20-30 | Head twitch |
|---|---|---|---|---|
| | Minutes | Minutes | Minutes | per rat |
| Control | 3 | 1 | 0 | 0.6 |
| MOC + DMT | 5 | 7 | 0 | 2 |
| MOC + Psilo | 9 | 13 | 9 | 5.16 |

### Example 11—Assessment of 5-HT₂ Receptor Activation

In murine studies, activation of 5-HT_{2A} receptors can be assessed by observing the head-twitch response (HTR) in treated animals. The head-twitch response is a rhythmic paroxysmal rotational head movement that occurs in mice and rats following 5-HT_{2A} receptor activation (see Halberstadt A L et al. Multiple receptors contribute to the behavioral effects of indoleamine hallucinogens. Neuropharmacology. 2011; 61:364-381).

Two methods are currently used to assess the HTR in laboratory studies. Most commonly, a trained rater observes the animals directly and counts the number of head twitches. Alternatively, the behaviour of the test animals may be video recorded for off-line assessment (see Canal C E, et al. The serotonin 2C receptor potently modulates the head-twitch response in mice induced by a phenethylamine hallucinogen. Psychopharmacology. 2010; 209:163-174; and Halberstadt A L, et al. Differential contributions of serotonin receptors to the behavioural effects of indoleamine hallucinogens in mice. Journal of Psychopharmacology. 2011; 25:1548-1561).

Various combinations of Moclobemide and DMT can be administered to mice or rats followed by assessment with the HTR indicating 5-HT_{2A} receptor engagement.

### Example 12—Human Studies

### i) Subjects

Eligible participate in a clinical study can be selected based on an appropriate age profile (e.g., 25 years and older) and with no previous experience or exposure to serotonergic psychedelic substances. All subjects can be screened to ensure they are physically healthy with appropriate BMIs and cardiovascular health.

Subjects with a current or past diagnosis of a mental health disorder as defined by the American Psychiatric Association Diagnostic and Statistical Manual of Mental Disorders, fifth edition (DSM-5), a history of suicide attempts, or any first- or second-degree relative with a psychotic or bipolar disorder may be excluded from the study.

### ii) Study Design

A randomised, double blind, placebo-controlled trial can be performed with the various dosages of DMT (such as: 2 mg, 4 mg, 8 mg, 16 mg, 32 mg, 64 mg, or 120 mg) or various doses of psilocybin (such as 100 μg, 250 μg, 500 μg, 1 mg, 1.5 mg, 2 mg, 4 mg or 6 mg) and various dosages of Moclobemide (such as 75 mg, 150 mg or 300 mg)-combined into a single oral dosage form with delayed release DMT. The formulation of the single oral dosage form can be varied to control the timing of the release of DMT with release kinetics of time of C₅₀ 30 minutes, 60 minutes, 90 minutes, 120 minutes or 150 minutes after administration.

At the screening visit, subjects would be required to provide written informed consent and psychiatric assessment (for example using the Mini-International Neuropsychiatric Interview). Subjects would be advised throughout the trial on what to expect and how to respond to the psychedelic experience.

Subjects may be admitted to an inpatient facility prior to and for the period of assessment. Healthcare professionals, including a therapist and a psychiatrist may be present in the room during treatment, with additional clinical staff supervising administration and sampling. Following treatment, subjects may be required to remain overnight for further psychological and safety evaluations, prior to discharged the next morning. Follow-up assessments can be conducted, in person, by phone, or via video call at set time intervals for up to 3 months.

iii) Pharmacokinetic Analysis

Blood samples can be taken via cannula prior to administration followed by set intervals (such as 5, 15, 30, 45, 90, 100, 110, 120, 130, 140, 150, 160, 170, 180, 240, 300, 360, 420, 480, 1380 min after administration). In addition, urine samples can be collected at various time points.

Plasma concentrations of Moclobemide and DMT at each timepoints can be analysed using known analysis techniques such as those described herein. The following PK parameters can be calculated: Maximum plasma concentration (Cₘₐₓ): time to peak plasma concentration (Tₘₐₓ); area under the plasma concentration-time curve from time zero to time of last measurable concentration (AUCₗₐₛₜ); area under the plasma concentration-time curve from time zero to infinity (AUCinf); terminal half-life (t_{1/2}); clearance (CL); apparent volume of distribution during terminal phase after IV administration (V_{z}); volume of distribution at steady state after IV administration (Vₛₛ); and mean residence time after IV administration (MRT_{inf}).

Pharmacokinetics can be determined using known statistics and PK modelling analysis.

### Example 13—Assessment of Experience

Assessment of psychedelic experiences or hallucinations were determined via known and recognised tests including the Hallucinogen Rating Scale (HRS) (Strassman et al. 1994) and the five-dimensional altered states of consciousness (5D-ASC) assessment (Dittrich A, et al. 5D-ASC Questionnaire for the Assessment of Altered States of Consciousness: A Short Introduction, Psychologisches Institut fur Beratung und Forschung, Zurich, Switzerland (2006); and Studerus et al. Psychometric evaluation of the altered states of consciousness rating scale (OAV). PLOS One. 2010; 5: e12412).

Additional tests include the Ego Dissolution Inventory (EDI), the Satisfaction with Life Scale (SWL), the Depression, Anxiety, Stress Scale-21 (DASS-21), the Five Facets Mindfulness Questionnaire (FFMQ-15), and the Brief Symptom Inventory-18 (BSI-18) (see Nour M M et al. Ego-dissolution and psychedelics: validation of the Ego-dissolution inventory (EDI). Frontiers in Human Neuroscience. 2016; 10:269; Diener E et al. The satisfaction with life scale. The Journal of Personal Assessment. 1985; 49:71-75; Henry J D and Crawford J R. The short-form version of the Depression Anxiety Stress Scales (DASS-21): construct validity and normative data in a large non-clinical sample. British Journal of Clinical Psychology. 2005; 44:227-239; Baer R A et al. Using self-report assessment methods to explore facets of mindfulness. Assessment. 2006; 13:27-45; and Derogatis L R (2001) Brief symptom inventory 18. Johns Hopkins University, Johns Hopkins University).

One or more of the above tests were utilised prior to and at various time points during or subsequent to treatment to gauge the experience of the participants. Statistical analysis was performed using techniques known in the art.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be further understood that terminology such as “comprise”, or variations such as “comprises” or “comprising” inherently include within their scope (without being limited to) versions of the invention that excludes other elements directly related to the invention. Accordingly, terminology such as “consisting of” or “consisting essentially of” can be substituted for terminology such as “comprise”, “comprises” or “comprising” with the effect of limiting the scope of the invention to the specifically recited elements. Notably, where it is explicitly intended for the invention to be considered in an exhaustive manner, such limitations should be considered to relate only to the inventive concept disclosed herein and other features can be added which fall outside of the scope of the inventive concept. Such features or elements may include, but are not limited to, excipients, formulations, additives, diluents, packaging, adjuvants and collocated features which are not to be excluded by terminology such as “consisting of” or “consisting essentially of”.

Referenced documents, publications and patents are to be included in their entirety by way of reference. The teachings and disclosures in such documents, publications and patents are therefore considered to form part of the disclosure of this specification.

All methods described herein can be performed in any suitable order unless indicated otherwise herein or clearly contradicted by context. The use of any and all examples, or exemplary language (e.g., “such as”, “i.e.”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

It will be apparent to the person skilled in the art that while the invention is described herein in detail for the purposes of clarity and understanding, various modifications and alterations to the embodiments and methods described herein may be made without departing from the scope of the inventive concept disclosed in this specification.

1-4. (canceled)

5. A method of treating a neurodevelopmental, neurological, movement, psychological or physiological, disease, disorder, syndrome or symptom in a human subject, the method comprising orally administering a single dosage form composition comprising a monoamine oxidase inhibitor (MAOI) and a tryptamine, wherein the MAOI is formulated for immediate release and the tryptamine is formulated for delayed release, and wherein the tryptamine is a substrate for a monoamine oxidase (MAO) enzyme.

6. The method according to claim 5, wherein the MAOI is moclobemide.

7. The method according to claim 5, wherein the tryptamine is a psychedelic tryptamine.

8. The method according to claim 5, wherein the tryptamine is selected from the group consisting of psilocin, a prodrug of psilocin, psylocibin, N,N-dimethyltryptamine (DMT), 5-methoxy-N,N-dimethyltryptamine (5-MeO-DMT) and pharmaceutically acceptable salts thereof.

9. The method according to claim 5, wherein the tryptamine is N,N-dimethyltryptamine, psilocybin, psilocin or a pharmaceutically acceptable salt thereof.

10. The method according to claim 5, wherein release of the MAOI is complete within about 1 hour.

11. The method according to claim 5, wherein the release of the tryptamine occurs over a time of between about 2 hours up to 12 hours after administration.

12. The method according to claim 5, wherein about 50% of the tryptamine is released between about 2 hours and about 4 hours after administration and about 90% of the psychedelic tryptamine is released between about 8 hours and about 10 hours after administration.

13. A single dose composition for oral delivery to a human subject comprising a monoamine oxidase inhibitor (MAOI) and a tryptamine, wherein the MAOI is formulated for immediate release and the tryptamine is formulated for delayed release, and wherein the tryptamine is a substrate for a MAO.

14. The composition according to claim 13, wherein the MAOI is moclobemide.

15. The composition according to claim 13, wherein the tryptamine is a psychedelic tryptamine.

16. The composition according to claim 13, wherein the tryptamine is selected from the group consisting of psilocin, a prodrug of psilocin, psylocibin, N,N-dimethyltryptamine (DMT), 5-methoxy-N,N-dimethyltryptamine (5-MeO-DMT) and pharmaceutically acceptable salts and prodrugs thereof.

17. The composition according to claim 13, wherein the tryptamine is selected from N,N-dimethyltryptamine (DMT), psilocybin, psilocin or a pharmaceutically acceptable salt thereof.

18-20. (canceled)

21. The composition according to claim 14, wherein moclobemide is present in an amount of between about 25 mg and about 1200 mg.

22. The composition according to claim 13, wherein the release of the MAOI is complete within about 1 hour after administration.

23-26. (canceled)

27. The composition according to claim 13, wherein release of the tryptamine is delayed for about 2 hours after administration.

28. (canceled)

29. The composition according to claim 13, wherein about 50% of the tryptamine is released between about 2 hours and about 4 hours after administration and about 90% of the tryptamine is released between about 8 hours and about 10 hours after administration.

30-35. (canceled)

36. The method according to claim 5, wherein the MAOI is selected from the group consisting of 1,2,3,4-tetrahydro-ß-carboline, 1,2,3,4-tetrahydroisoquinoline (TIQ), 2-methyl-1,2,3,4-tetrahydro-ß-carboline, Amiflamine, Befloxatone, Benmoxin, Bifemelane, Brofaromine, Caroxazone, Clorgyline, Curcumin, CX157 (TriRima), Epicatechin, Eprobemide, Esuprone, Harmaline, Harmane, Harmine, Hydracarbazine, Iproclozide, Iproniazid, Isocarboxazid, Mebanazine, Methylene blue, Methylthioninium chloride, Metralindole, Minaprine, Moclobemide, Nialamide, Norharmane, Octamoxin, Pargyline, Phenelzine, Pheniprazine, Phenoxypropazine, Pirlindole, Pivalylbenzhydrazine, Procabazine, Procyanidins, Quercetin, Rasagiline, Rosiridin, Safinamide, Safrazine, Sercloremine, Selegiline, Tetrahydroharmine, Tetrindole, Toloxatone, and Tranylcypromine.

37. A method of treating a neurodevelopmental, neurological, movement, psychological or physiological, disease, disorder, syndrome or symptom in a human subject, the method comprising orally administering a single dosage form composition comprising a monoamine oxidase inhibitor (MAOI) which is moclobemide and a tryptamine which is N,N-dimethyltryptamine (DMT), wherein the MAOI is formulated for immediate release and the tryptamine is formulated for delayed release, and wherein the tryptamine is a substrate for a monoamine oxidase (MAO) enzyme,
wherein release of the MAOI is complete within about 1 hour, and release of the tryptamine is delayed for about 2 hours and then occurs over a time of up to 12 hours, following administration.

38. The composition according to claim 13, wherein the monoamine oxidase inhibitor (MAOI) is moclobemide present in an amount of between about 25 mg and about 1200 mg, and the tryptamine is N,N-dimethyltryptamine (DMT), and
wherein release of the MAOI is complete within about 1 hour, and release of the tryptamine occurs over a time of up to 12 hours but is delayed for about 2 hours, following administration.
